(19) United States
(12) Patent Application Publication
(10) Pub. No.: US 2011/0183891 A1
(43) Pub. Date: Jul. 28, 2011

(54) Methods and Compositions for Inhibiting Fungal Infection and Disease

(76) Inventors: Jordan Tang, Edmond, OK (US); Hao Wu, Oklahoma City, OK (US)

(21) Appl. No.: 12/967,896

(22) Filed: Dec. 14, 2010

Related U.S. Application Data
(60) Provisional application No. 61/287,074, filed on Dec. 16, 2009.

(51) Int. Cl. A61K 38/06; C07K 5/00; C07K 14/00; A61K 38/16; A61K 38/08; A61K 38/07; A61K 38/12; C07K 7/64; A61P 31/00; C12N 9/99

(52) U.S. Cl. 514/3.4; 530/330; 530/326; 514/3.3; 530/317; 435/184

(57) ABSTRACT

The present invention describes a previously unknown interaction between secreted aspartic proteases (SAPs), including SAPs 4-6 of Candida albicans, and integrins on host cells. The SAPs secure entry into the host cell through RGD-like binding motifs and subsequently induce apoptosis, thereby clearing the way for systemic infection. The invention thus provide a new target for therapeutic intervention and describes peptides and antibodies that inhibit the action of SAPs in this context, including their interaction with integrins.

The present application claims benefit of priority to U.S. Provisional Application Ser. No. 61/287,074, filed Dec. 16, 2009, the entire contents of which are hereby incorporated by reference.

### BACKGROUND OF THE INVENTION

1. Field of the Invention

The present invention relates generally to the fields of fungal disease and methods of treating the same. More particularly, it concerns unique agents that target fungal invasion processes.

2. Description of Related Art

The AIDS epidemic, advances in surgical procedures, and aggressive anti-cancer therapy have contributed to the surge of immunocompromised populations. Coinciding with this surge is an increase in the incidence of clinically significant fungal infections (Dixon et al., 1996; Henderson and Hirvela, 1996). Candida albicans has become the fourth leading cause of nosocomial infections, with systemic candidiasis having a very high mortality rate, especially in newborns—up to 65% (Pacheco-Rias et al., 1997), and among cardiac surgery patients—up to 30% (Michaloupoulos et al., 1997). The majority of AIDS patients experience some form of candidiasis and many have to take antifungal drugs repeatedly, or even prophylactically on a daily basis. In the healthy population, more than half of all women experience at least one vaginal yeast infection, and about 8% suffer recurrent episodes. The morbidity, mortality and health care costs associated with fungal infections has commanded a need for effective antifungal agents.

Only a few classes of antifungal drugs are actively used in clinics. Flucytosine, a substituted pyrimidine, is converted by a fungi-specific cytosine deaminase into 5-fluorouracil which causes the inhibition of DNA and protein synthesis. Due to frequent emergence of resistance, flucytosine is rarely used alone and is often co-administered with amphotericin B (Alexander & Perfect, 1997).

Amphotericin, a polyene antibiotic, has the broadest spectrum of activity of any available antifungal agent and is fungicidal when tested in vitro. It interacts with membrane sterols, alters membrane permeability and causes membrane leakage and death of the pathogen. However, amphotericin is toxic and has a very narrow therapeutic index. Even in therapeutic doses, it often causes severe side effects, including fevers, chills, nausea, vomiting, and nephrotoxicity (Brajtburg and Bolard, 1996).

Azole drugs, such as fluconazole, ketoconazole, and itraconazole, are much less toxic and have become drugs of choice for most indications. The primary target of azoles is the heme protein, lanosterol 14α-demethylase. By inhibiting this enzyme azoles prevent the synthesis of the major sterol of the fungal membrane, ergosterol, and cause accumulation of intermediate products (Kauffman and Carver, 1997).

The degree of the damage to fungal cells caused by the alterations of membrane sterols depends on the nature of the pathogen. While highly effective against Saccharomyces cerevisiae, azoles are less detrimental to Candida. They are not fungicidal toward the most common human fungal pathogen, C. albicans, and even their inhibitory effect on the growth of this yeast differs widely among different fungal isolates. While the growth of some isolates is strongly inhibited, the majority continue to grow even at very high concentrations of the drug with completely depleted ergosterol. This so-called post-MIC growth creates significant difficulties in determining the azole sensitivity of C. albicans isolates in clinical laboratories. While the standards for determining minimal inhibitory concentrations (MIC) of most antimicrobial agents define MIC as the lowest concentration of the drug preventing any visible growth of a pathogen, the NCCLS standard for antifungal susceptibility testing (document M27-A) had to be formulated much less strictly and defines MIC as the lowest concentration of a drug causing 80% growth inhibition (NCCLS, 1997). The moderate inhibitory effect of azoles on the growth of C. albicans is also reflected in the phenomenon of “trailing endpoint” when the apparent MIC, or more correctly MIC₈₀, determined in broth microdilution tests shifts during the incubation (Rex et al., 1996; Revankar et al., 1998a). For most isolates the MIC of fluconazole lies below the clinically achievable 4 μg/ml if determined after 24 hours of incubation, but for many of them it exceeds 64 μg/ml after 48 hours.

The clinical effectiveness of azoles against C. albicans clearly exceeds their in vitro effectiveness. Indeed, isolates exhibiting a high rate of post-MIC growth in vitro were obtained from patients whose fungal infections were in fact later successfully treated with azole drugs (Revankar et al., 1998b). The reason for this discrepancy is that in the organism of a patient fungal infections are being suppressed not only by drug therapy but also by host defense mechanisms including phagocytes and antifungal immune response. Although merely slowing down the growth of the pathogen, azole drugs make it more susceptible to host defenses. Besides simply changing the dynamics of infection through growth inhibition, azoles have also been reported to make C. albicans cells more susceptible to phagocytes (De Brabander et al., 1980; Shigematsu et al., 1981).

In spite of the relative clinical success of azole drugs as compared to other antifungal agents, their inability to kill Candida cells without relying on host defense mechanisms is the likely reason for two highly undesirable clinical outcomes: recurrence of infection and development of azole resistance. As mentioned above, a significant percentage of women are suffering from recurrent vaginitis. In these cases azoles alleviate symptoms of infection but the infection relapses again a short time after treatment. The relapsed strain usually has the same sensitivity to the drug as the initial one (Fong et al., 1993; Lynch et al., 1996), thus suggesting that azole resistance is not the underlying cause of recurrence. Such host factors as immune deficiency, allergy, use of contraceptives, local pH, deficient production of IgA antibodies, and even psychological factors have been implicated in the phenomenon of recurrent vaginitis (White et al., 1997; Blasi et al., 1998; Irving et al., 1998; Kubota, 1998; Clancy et al., 1999). Importantly, however, molecular fingerprinting of the pathogen genome has shown that in more than 80% of cases the C. albicans strain which causes relapse is the same strain that caused initial infection (Schroppel et al., 1994; Fong, 1994, Vazquez et al., 1994; Lockhart et al., 1996). Similarly, recurrent azole-treated oropharyngial candidiasis which affects 50% of AIDS patients has been associated with re-growth of the same strain of C. albicans rather than with reinfection with other strains or development of azole resistance (Boerlin et al., 1996). It is highly likely, therefore, that many cases of recurrent candidiasis could have been prevented if azole drugs eradicated yeast cells rather than merely inhibited their growth.

Besides dramatically increasing the chances for the recurrence of infections, the survival of azole-treated Candida cells creates a breeding ground for the development of azole resistance. This resistance has become a serious clinical problem in recent years: its incidence is on the rise (Cameron et al., 1993; Redding et al., 1994; Revankar et al., 1998b), which endangers the future use of azole drugs in clinics. Clinical isolates of C. albicans demonstrate a number of biochemical mechanisms of resistance (reviewed in Sanglard et al., 1995; White et al., 1998; Vanden Bossche et al., 1998). The first group of these mechanisms deals directly with the target of azole action, lanosterol 14α-demethylase. Point mutations in the gene of this enzyme, ERG11, alternatively called ERG16 or CYP51, over expression of this gene, or its amplification have been described in resistant clinical isolates of C. albicans. Additionally, azole-resistant C. albicans have been shown to over express multidrug efflux pumps: CDR1, CDR2, and MDR1. Expression of these membrane proteins leads to the decrease in the accumulation of azole drugs in the yeast cytoplasm and thus reduces their antifungal activity.

Importantly, each of these mechanisms individually provides relatively low level of azole resistance. Clinically resistant strains usually display a combination of resistance mechanisms described above. The development of these strains is a multi-step process in which genetic changes leading to resistance are accumulating gradually in response to selection with drugs (White, 1997; Franz et al., 1998; Franz et al., 1999; Lopez-Ribot et al., 1998; Cowen et al., 2000). The inability of azoles to kill yeast cells promotes this process. Indeed, a mutation leading to even a minor increase in the MIC of the drug gives mutated cells selective advantage over parental cells, so that they gradually overcome the yeast population infecting the patient. If azoles were fungicidal, both the parental cells and the cells with a slightly increased azole MIC would be eliminated, thus dramatically reducing chances for the development of resistant strains.

In summary, the clinical success of azole therapy of C. albicans infections is limited by the rather moderate inhibitory effect of ergosterol depletion on this pathogen. Large pharmaceutical companies are tying to improve the effectiveness of antifungal therapy by identifying alternative drugs attacking new molecular targets of the pathogen. As of yet, these extensive screening programs have not yielded a drug with an activity significantly exceeding that of azoles. An alternative approach to drug discovery has been utilized previously by the inventors, namely, the identification of potentiators of existing antimicrobial agents. In particular, in this bacterial work, the inventors have identified a number of potentiators of fluoroquinolone antibiotics, which act by inhibiting multidrug-efflux transporters of pathogenic Gram-positive cocci (Markham et al., 1999). More recently the inventors identified compounds which, when combined with bacteriostatic antibiotics, exert bactericidal effect. With respect to antifungal agents, the inventors embarked on finding a compound that would potentiate the antifungal effect of azoles, the most effective and popular antifungal drugs to date.

### SUMMARY OF THE INVENTION

Thus, in accordance with the present invention, there is provided a method of inhibiting a secreted aspartic protease (SAP) cleavage of a target substrate comprising contacting said SAP with a peptide comprising at least four residues and having the formula:

P₂-P₁-P_{1′}-P_{2′}

wherein P₁, P₂, and P_{1′}, can be any residue, and P_{2′} is a negatively-charged residue. The peptide may be 4-25 residues in length. The P_{2′} negatively-charged residue may be aspartic acid, glutamic acid, phosphoric acid or sulfonic acid. The peptide may comprise the sequence:

P₂-P₁-*-P_{1′}-P_{2′}

wherein -*- indicates modification of the peptide bond into a transition state analog. The peptide may comprise the sequence SHLPS(E/D)FT or SHLP*S(E/D)FT. The peptide may comprise an XGY motif, wherein X is positively-charged residue, and Y is a negatively-charged residue. The peptide may comprise the sequence RGD-SHLPS(E/D)FT or SHLPS(E/D)FT-RGD, or SHLP*S(E/D)FT or SHLP*S(E/D)FT-RGD, wherein * indicates modification of the peptide bond into a transition state analog.

The SAP may be SAP4, SAP5 or SAP6, or may be a pathogen SAP, such as yeast or fungus, including but not limited to a Candida species (C. albicans, Candida tropicalis, Candida dubliniensis, Candida glabrata) or Aspergillus species.

In another embodiment, there is provided a peptide comprising at least four residues and having the formula:

P₂-P₁-*-P_{1′}-P_{2′}

wherein P₁, P₂ and P_{1′} can be any residue, and P_{2′} is a negatively-charged residue, and -*- indicates modification of the peptide bond into a transition state analog. The peptide may be 4-25 residues in length. The P_{2′} negatively-charged residue may be aspartic acid, glutamic acid, phosphoric acid or sulfonic acid. The peptide may comprise the sequence SHLP*S(E/D)FT. The peptide may further comprise an XGY motif, wherein X is a positively-charged residue, and Y is a negatively-charged residue. The peptide may comprise the sequence RGD-SHLP*S(E/D)FT or SHLP*S(E/D)FT-RGD. The peptide may be linked to Integrilin®, to another drug such as an anti-fungal agent or a transition state inhibitor.

In still yet another embodiment, there is provided a method of inhibiting a fungal infection in a subject comprising administering to said subject a XGY motif peptide, wherein X is a positively-charged residue, and Y is a negatively-charged residue. The peptide may be 4-25 residues in length. The XGY motif peptide may be linked to a second peptide having the formula:

P₂-P₁-*-P_{1′}-P_{2′}

wherein P₁, P₂, and P_{1′} can be any residue, and P_{2′} is a negatively charged residue, and -*- indicates modification of the peptide bond into a transition state analog. The P_{2′} negatively-charged residue may be aspartic acid, glutamic acid, phosphoric acid or sulfonic acid. The second peptide may comprise the sequence SHLP*S(E/D)FT. The fungal infection may be caused by a Candida species or Aspergillus species. The XGY motif may comprise RGD or RGDS. The XGY motif peptide may be comprised in Integrilin®. The subject may be a human subject. The peptide may be linked to an anti-fungal agent.

Also provided is a method of inhibiting a fungal infection in a subject comprising administering to said subject an antibody that binds immunologically to an XGY motif in a secreted aspartic protease, wherein X is a positively-charged residue, and Y is a negatively-charged residue. The motif may be RGD or RGDS. The fungal infection may be caused by a Candida species or Aspergillus species.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” “About” is defined as including amounts varying from those stated by 5-10%.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—(FIG. 1A) Sequence conservation of SAP 4-6 subfamily from C. albicans. Alignment sequences of SAP 4 (GI: 3640365), SAP 5 (GI: 3639268) and SAP 6 (GI: 3639094) are from C. albicans. Sequence of 3PEP is from the porcine pepsin (GI: 157836865). Highly conserved RGD motif is in the rectangle. There are three ‘arms” are marked by cyan arrows. (FIG. 1B) The highly conserved residues are shown in BOLD type. The residues of SAPs in the Arm A area are indicated by horizontal bar. In all three SAPs, integrin binding motif RGD (underlined) is found in Arm A.

FIG. 2—3D structure of SAP 5 from Subfamily of C. albicans. The “RGDKGD” motif is located at the top portion of the figure underneath the label. The pepstatin A (IHN) is located in the active sites. The conserved motif of “YYT” in subfamily SAP 4-6 is located at the top right and is labeled “*”. The amino acids located at the top right and labeled “+” are the “DXXG” motif, which functionally binds to Mg²⁺ in GTPase superfamily. The figure was generated with Pymol. PDB ID code: 2qzx.

FIGS. 3A-B—Human platelets bind SAP 6 from C. albicans. (FIG. 3A) Fluorescence microscope imaging of SAP 6 binds to human platelets. The white arrows in the fluorescence imaging, transmit imaging and the merge imaging show this platelet was bound with Alexa Fluor® 488 labeling SAP 6. (FIG. 3B). Confocal imaging by the deconvolution software. The white arrow shows that SAP 6 binds to the perimeter of this platelet based on the cross sections of the images after deconvolution.

FIGS. 4A-B—ADP activates SAP 6 binding to human platelet and the binding is dose-dependent inhibition by RGDS peptide and Integrilin®. Cells were incubated with labeled enzyme together with ADP and inhibitors respectively at 40° C., then pellets were resuspended in 100 ml Hepes-Tyrode buffer, and fluorescence measured by TECAN (Ex./Em.=488 nm/519 nm). (FIG. 4A). Dose-dependent inhibition of SAP 6 binding to human platelets (FIG. 4B). The inhibition of RGDS peptide is much stronger than that of Integrilin®. T test (mean with SEM), n=3, *P<0.05, **P<0.001.

FIGS. 5A-E—Inhibition of attachment of labeled Alexa Fluoro®-488 SAP 6 by RGDS peptide and Integrilin®. (FIG. 5A) Cells only (control): the ratio of positive cells in R4 is 0.69%; (FIG. 5B) Cells+Labeled SAP 6: the ratio of positive cells in R4 is 3.23%; (FIG. 5C) Cells+400 μM Integrilin®+Labeled SAP 6: the ratio of positive cells in R4 is 2.42%; (FIG. 5D) Cells+400 μM RGDS+Labeled SAP 6: the ratio of positive cells in R4 is 1.68%. Therefore, RGDS and Integrilin® significantly inhibit SAP 6 binds to human lung A549 cells. (FIG. 5E) Representative data of histogram of the inhibition of SAP 6 binding to human carcinoma lung cells A549.

FIG. 6—Relative binding anti-131 antibody to A549 cells. Anti-Cells+Buffer+anti-β1 antibody. (T test, mean with SD, N=3, **p<0.01).

FIG. 7—Specific inhibition assay of SAP 6 binding to A 549 cell. RDGRG is less inhibitory of SAP 6 binding to A549 cells compared with that of RGDS. This means that SAP 6 binds to human lung carcinoma cells via RGD motif.

FIGS. 8A-D—The initial binding (10° C.) and later endocytosis (37° C.) assay of SAP 6 to Human Lung carcinoma Cells A549 assessed by confocal microscopy. FIGS. 8A and 8C show SAP 6-Alexa Fluoro® 488 added to A549 cells and incubated at 10° C. for 30 min in the Lab-Tek® II chamber slide, then rinsed with completed growth medium without phenol red for three times, followed by detection with confocal microscopy. FIGS. 8B and 8D show SAP 6-Alexa Fluoro®-488 added to A549 cells, incubated at 37° C. for 30 min to 1 h in the Lab-Tek® II chamber slide, followed by detection using fluorescence intensity. Based on this data, SAP 6 can induce the endocytosis at physiology conditions (37° C.).

FIG. 9—SAP 6 binding to integrin of A459 cells. SAP 6-Alexa Fluoro® 647 incubated A549 cells at 37° C.

FIG. 10—Nomenclature on the subsites of protease substrates. A hypothetical peptide substrate with the sequence of Glu-Val-Asn-Leu-Ala-Ala-Glu-Phe is shown here. The protease cleavage site (arrow) is between Leu and Ala. The residues on the left (toward the N-terminus) of the cleavage position are named P1, P2, P3 and P4, while those on the right (toward the C-terminus) are named P1′, P2, and P3′ and P4′. The corresponding binding sites on the proteases are called S1, S2, S3 etc. with the letter S substitute the letter P.

FIG. 11—Hydrolysis of globin from bovine hemoglobin by SAP 4 (left), SAP 5 (center) and SAP 6 (right). The proteases were separately incubated with globin then mixed with trichloroacetic acid to 1.25%. The globin fragments resulted from protease digestion were TCA soluble and were quantitated by OD at 280 nm in a spectrophotometer. The plots show that the increase of amount of enzyme in each case resulted in the increase of digestion products.

FIG. 12—Amino acid sequences around the cleavage sites (vertical line) of bovine hemoglobin by C. albicans SAP 4-6. The amino acid residues are shown in single-letter codes. Preference of an acidic amino acid, either aspartic acid (D) or glutamic acid (E), at P2′ subsite is shown in red. No clear preference is seen in other subsite positions among eight residues, from P4 to P4′, usually recognized by aspartic proteases.

FIG. 13—Cell death assay by trypan blue method. Kinetic assay of cell killing of SAP 2 and SAP 6 by Trypan Blue. SAP 2 and SAP 6 were incubated with the same amount of A549 cells at 37° C. for 25 h. SAP 6 killed epithelial cells much faster than SAP 2.

FIG. 14—Apoptosis triggered by C. albicans SAPs. Sample I: Cells+SAP 6-Alexa Fluoro® 488; Sample II: Cells+0.4 mM Integrilin®+SAP 6-Alexa Fluoro® 488; Sample III: Cells+RGDS+SAP 6-Alexa Fluoro® 488. After cells sorting by FACS, the samples were incubated at 37° C. for 4 h, then counted by Trypan Blue. The stained cells (dead cells) of fluorescence labeled cells were more than that of non-fluorescence labeled cells. Fluorescence cells were the cells which bound with labeled SAP 6; non-fluorescent cells were the cells which did not bind with labeled SAP 6.

FIGS. 15A-B—Early apoptosis of A549 induced by C. albicans SAPs. (FIG. 15A) The same amount A549 cells (−55×10⁴ Cells/ml) was incubated with 1 mM SAP 2 and SAP 6 respectively for ˜10 hrs, then flow cytometry was performed. SAP 6 induced early apoptosis of A549 more significantly than SAP 2. (FIG. 15B) The same amount A549 cells (˜55×10⁴ Cells/ml) was incubated with 1 mM SAP 2, SAP 4-6 respectively for ˜10 hrs, then flow cytometry was performed. SAP 4 and SAP 6 induced early apoptosis of A549 more significantly than SAP 2. SAP 5 also induced early apoptosis. Data are expressed as mean±SEM; N=3; *p<0.05.

FIGS. 16A-B—C. albicans SAPs induced apoptosis of A549 cells. (FIG. 16A) The early apoptosis of A549 induced by mixed different SAPs. After incubating A549 at 37° C. for 6 hours with SAP 2 and SAPs 4-6, the same amount of SAP 6 was added into the SAP 2 sample and SAP 2 was added into SAP 4-6 samples, and continually incubated at 37° C. for another ˜10 hrs. Flow cytometry was performed to detect intensity of PE Annexin V of A549. The data significantly shows that the early apoptosis of A549 treated with SAP 4-6 following added SAP 2 is higher than that of A549 treated with SAP 2 following added SAP 6. (FIG. 16B) The same experiment as FIG. 16A was performed. The mixtures of samples were as follows: full—added 60 μl enzymes at the very beginning and incubated at 37° C. for ˜16 h; partial delay—added half the amount of enzymes (30 μl) at the very beginning, after incubated at 37° C. for 6 h, then added another half of amount of the same enzymes (30 μl), and continually incubated at 37° C. for ˜10 h; partial delay & mixture—added half the amount of enzymes (30 μl) at the very beginning, after incubated at 37° C. for 6 h, then added another half of amount of the different enzymes (30 μl), and continually incubated at 37° C. for ˜10 h. The early apoptosis of partial delay and mixture of SAP 4-6+SAP 2 is significantly higher than those of partial delay of SAP 4-6+SAP 4-6. Data are expressed as mean±SEM; N=3; *p<0.05.

FIG. 17—LMP induced by SAP 2 and SAP 6. 200 μl cells were seeded with 23×10⁴ cells/ml in sterile chamber plate (Willco wells BV-WG PLEIN 275) and incubated at 37° C. for 7 h. 100 μl buffer (10 mM HEPES pH 7.0, 150 mM NaCl) was added to 0.28 mg/ml SAP 6 and 0.28 mg/ml SAP 2 in the chamber, and incubated at 37° C. for 24 h. Quantification of red (left three bars) and green (right three bars) fluorescence intensity (randomly chose 3˜6 regions; n=3, Mean+SD).

FIG. 18—LMP induced by combination of SAP 2 and SAP 6. 200 μl cells were seeded with 36×10⁴ cells/ml in sterile chamber plate incubated at 37° C. for 3.5 h. 80 buffer (10 mM HEPES pH 7.0, 150 mM NaCl) was added to 0.28 mg/ml SAP 6 and 0.28 mg/ml SAP 2 in different samples, respectively, and incubated at 37° C. After incubation for 4 h, another 80 μl SAP 6 or 80 μl SAP 2 was added into the relative samples which had already received SAP 2 or SAP 6, respectively, and these were continually incubated at 37° C. for another ˜10 h. After rinsing the cells with 1×PBS three times, 5 μg/ml Acridine Orange was added and the cells incubated at 37° C. for 15 min. The cells were rinsed with 1×PBS for three times. Acridine Orange relocalization was detected by a Zeiss LSM LIVE DUO confocal system. Quantification of red (left bar of each pair) and green (right bar of each pair) fluorescence intensity (randomly chosen 3˜6 regions; n=3, Mean+SD).

FIGS. 19A-B—The inhibition of LMP of A 49 by synthetic inhibitor of GRL-001-10CAND. The synthetic inhibitor of GRL-001-10CAND is not good inhibitor for SAP 5 and SAP 6. (FIG. 19A) Quantification of red (left four bars) and green (right four bars) fluorescence intensity. (FIG. 19B) Quantification of red (left pair of bars) and green (right pair of bars) fluorescence intensity induced by SAP 6. GRL-001-10CAND cannot inhibit LMP induced by SAP 6 (confocal imaging not shown). Randomly chose 3-6 regions; n=3, Mean+SD.

### DETAILED DESCRIPTION OF THE INVENTION

As discussed above, candidiasis is an infection caused by Candida fungi, especially Candida albicans. These fungi are found almost everywhere in the environment. Some may live harmlessly along with the abundant “native” species of bacteria that normally colonize the mouth, gastrointestinal tract and vagina. Usually, Candida is kept under control by the native bacteria and by the body's immune defenses. If the mix of native bacteria is changed by antibiotics, the body moisture that surrounds native bacteria can also have subtle changes in its acidity or chemistry. This can cause yeast to grow and to stick to surfaces, so that the yeast causes symptoms. Candida infections can cause occasional symptoms in healthy people. If a person's immune system is weakened by illness (especially AIDS or diabetes), malnutrition, or certain medications (corticosteroids or anti-cancer drugs), Candida fungi can cause symptoms more frequently. Candidiasis can affect many parts of the body, causing localized infections or larger illness, depending on the person and his or her general health. The frequency of infection by different strains of Candida is: Candida albicans, 57.8%; Candida tropicalis, 12.7%; Candida glabrata, 8.8%, Candida famata, 7.8% and other Candida spp., 12.9%. Therefore, Candida albicans is the most important Candida pathogen for the development of treatment of Candidiasis.

The present invention relates to the inventors' discovery that the RGD integrin binding motifs at the tip of a surface peptide strand on C. albicans SAPs 4, 5 and 6 are utilized to bind integrin, gain entrance to cellular interior and cause cell death, likely by triggering apoptosis. This is a newly discovered virulence mechanism, as it previously was thought that SAPs attack cell surface proteins from the outside to loosen up cell-to-cell associations, thus gaining entry and causing systemic infection. This new mechanism causes the death of epithelial or endothelial cells from the inside, thereby gaining entry for tissue invasion. Indeed, the hyphal form of C. albicans is known to be associated with invasiveness and is also the form that secrets SAPs 4-6. By exploiting the knowledge of this new specific target, the present inventor proposes to treat fungal infections using agents that interfere with SAP 4-6 function.

### I. FUNGI AND THEIR RELATED PATHOLOGIES

In the United States, blastomycosis, coccidioidomycosis and histoplasmosis are the major causes of systemic mycotic infection in normal human hosts. Sporotrichosis is a fourth invasive fungal disease, but occurs with broader distribution than the previous three. A variety of other fungal agents, including Candida and Aspergillus species, can colonize the mucocutaneous surfaces of normal human hosts, but rarely cause disease. Much more typical are fungal infections in immune-compromised individuals.

A. Blastomyces

Blastomycosis is a systemic mycotic infection that is cause by the dimorphic fungus Blastomyces dermatitidis. The initial portal of entry is the respiratory tract, with inhaled organisms deposited in the peripheral air spaces of the lower lobes. Hematogenous dissemination with metastatic spread to a variety of sites, particularly the skin, skeletal system, genitalia and central nervous system may occur. The pathologic hallmark is mixed acute and chronic inflammation. Treatment generally involves amphotericin B, given at a total dosage of 2.5 to 3.0 grams over 2 to 3 months. Fluconazole (400 mg/day) and itraconazole (400-800 mg/day) also have been employed more recently.

B. Histoplasma

Histoplasmosis, a systemic mycosis characterized by infection of the fixed and circulating phagocytic cells of the reticuloendothelial system, is caused by the dimorphic fungus Histoplasma capsulatum. The fungus grows in many parts of the world, particularly in soil enriched with the fecal material of birds or bats. Typical infection occurs when the soil is disturbed, cause aerosol infection. Regional spread to lymph nodes and bloodstream occurs rapidly. One to three weeks after infection, necrotizing granulomatous responses develop. Interferon-γ and IL-12 appear to be of great importance in defending from the disease. Typical treatment is with amphotericin B, in a total dose of between 500 and 1000 mg. Azoles also are suitable therapies.

C. Coccidioides

The causative agent for coccidioidomycosis is the dimorphic fungus Coccidioides immitis. It can exist as a non-invasive saprophyte on tissue surfaces, but inhalation of the arthrospores results in production of mature spherules, the definitive tissue pathogen. The natural habitat of the disease is in the lower Sonoran life zone, but transmission is so efficient, the disease may spread many miles away. In some endemic region, infection is virtually universal. Cell mediated immunity is critical to controlling the infection, and immune-suppressed individuals show reduced granuloma formation, and concomitant increase spherule burden. Amphotericin B, fluconazole and itraconazole all are used in treatment.

D. Sporothorix

Sporothorix schenckii is a dimorphic fungus found in both tropical and temperate climates. Disease commonly arises from subcutaneous inoculation with infections spores by a contaminated thorn or other sharp object. In rare cases, spores may be inhaled. Following subcutaneous implantation, pseudoepitheliomatous hyperplasia of the overlying layers of the skin develop, producing a verrucous, sometimes ulcerating lesion. From this initial site, there is slow spread along the draining lymphatics, and secondary skin lesions. Amphotericin B is the preferred treatment.

E. Candida

Candidiasis comprises clinical infections that are caused by different dimorphic fungi of the genus Candida. The most virulent are C. albicans and C. tropicalis, but C. krusei, C. parapsilosis and C. guilliermondii can cause disease in immunocompromised patients. Candida species are part of the normal GI flora in 50% of persons, and in vaginal flora in 20% of non-pregnant women. Overgrowth remains trivial unless the mucocutaneous surfaces are penetrated.

Variations on Candida pathology include mucosal candidiasis, cutaneous candidiasis, chronic mucocutaneous candidiasis, candidal peritonitis, candidal endocarditis, pulmonary candidiasis, urinary tract candidiasis, and disseminated candidiasis. Diagnosis is by microscopic examination and culture. Amphotericin B is the standard therapy, with a total dose of 500 to 1000 mg. Treatment typically involves mystatin, clotrimazole or miconazole for minor cutaneous or vaginal candidiasis. Fluconazole or itraconazole at 400 to 800 mg/day also may be used.

F. Aspergillus

Aspergillosis covers a group of different illnesses that have a major impact on the lungs, and are caused by dimorphic fungi of the genus Aspergillus. A single species, Aspergillus fumigatus, accounts for one-half to two-thirds or of all clinical disease caused by Aspergillus, with Aspergillus flavus accounting for most of the remainder. Aspergillus is almost always transmitted through the air, and it implants in the lungs, nasal sinuses, palate, and epiglottis. The most serious form of aspergillosis is found severely immunocompromised patients, characterized by necrotizing bronchopneumonia. Therapy usually involves amphotericin B, with possible surgical ablation. Flucytosine or rifampin often is added to the regimen. Azoles may be used as end stage “wrap-up” treatment.

G. Other

Other significant fungal infections are caused by Cryptococcus, Torulopsis, Paracoccidioides, Rhizopus, Mucor and Absidia species.

### II. SECRETED ASPARTIC PROTEASES

Human pathogenic fungi frequently cause infections of skin and mucosae; however, they are also capable of causing life threatening systemic mycoses. C. albicans is the most common fungal pathogen of humans and has become the fourth leading cause of nosocomial infection (Naglik et al., 2003; Naglik et al., 2008). At the most serious level, mortality rates from systemic candidiasis are high. However, the majority rates of patients, notably immuno-suppressed individuals with human immunodeficiency virus (HIV) infection, experience some form of superficial mucosal candidiasis, most commonly thrush, and many suffer from recurrent infection. The secreted aspartic protease (SAP) of Candida albican is the major virulence and opportunistic pathogen for these immune compromised people (Naglik et al., 2003).

There are total 10 SAPs in C. albicans. All 10 SAPs of C. albicans can be divided into subfamilies based on amino acid sequence homology alignments. They include SAP 1-3 (up to 67% identical), SAP 4-6 (up to 89% identical), and SAP 9-10 (C-terminal consensus sequences typical for GPI proteins). SAP 7 and SAP 8 are divergent and are not represented as subfamily members (Naglik et al., 2003; Naglik et al., 2008).

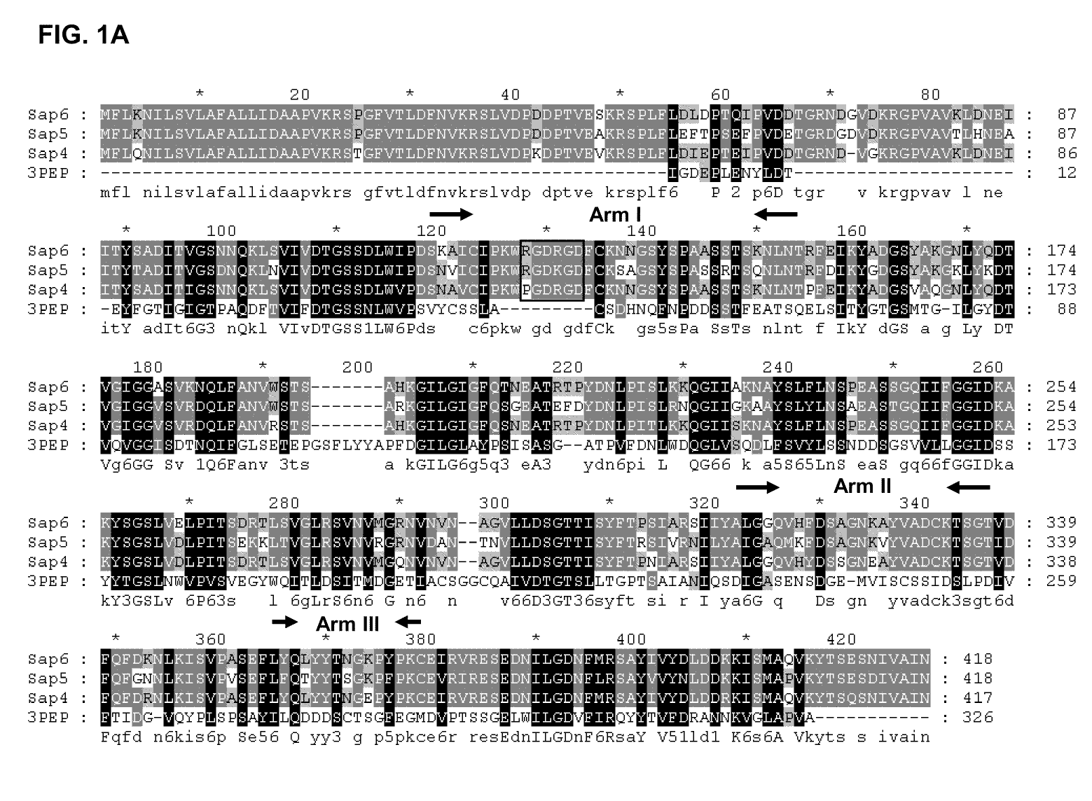

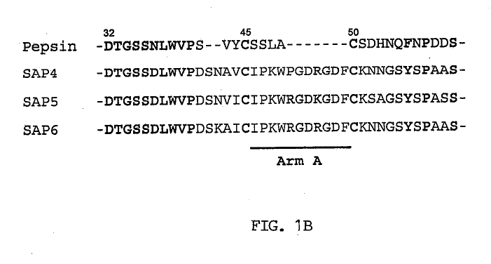

Most SAPs are secreted and have been demonstrated to be virulent factors for C. albicans infection. A comprehensive description on these proteases can be found in a review by Naglik et al. (2003). These 10 SAPs can be grouped according to their sequence homology as in FIG. 1. As can be seen, SAPs 1, 2 and 3 are a closely related. Similarly, SAPs 4, 5 and 6 are also closely related. This is in good agreement with the fact that SAPs 1, 2 and 3 have pH optima near pH 3.5 while SAPs 4, 5 and 6 have pH optima near pH 5.0 (Borg-von Zepelin et al., 1998). These relationships also predict that the SAPs in the same family may have similar functions in the pathogenesis of Candidiasis.

The deletion of the combination of several C. albicans SAPs rendered the loss of virulence in these mutants (see Naglik et al., 2003 for review), suggesting that the inhibition of the activity of SAP may be effective treatment for Candidiasis.

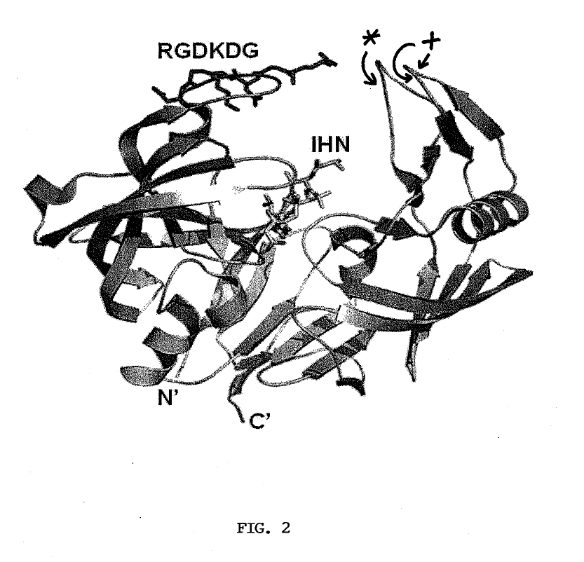

The crystal structures of four of the SAPs from C. albicans have been determined. Their 3-D structures are highly homologous to those of the pepsin family. Unique in the SAPs is the presence of three ‘arms’ extended above the structures of other aspartic proteases (as illustrated in the Arms A, B and C in FIG. 2). The functions of these arms are unknown.

In previous studies, SAPs 1-3 were shown to be specifically expressed in a particular switching phase of the yeast (opaque phase) (White and Agabian, 1995), and presumably play important roles during disseminated infections (Hube et al., 1997). SAP 2 is specifically activated in vitro when proteins are the sole nitrogen source. In vivo, SAP 2 is significantly activated in the late stages infection after spread to deep organs and concomitantly with tissue destruction (Staib et al., 2000; Staib et al., 1999). It seems that the SAP 2 proteinase may let C. albicans to thrive within the destroyed tissue by degrading host proteins for nutrient supply (Staib et al., 2000). Therefore, the critical role of a pioneer attacking the host during C. albicans infection is not performed by the SAP 1-3 subfamily. The precise mechanisms by which SAP proteinases contribute to the initial adherence process are not clear.

Dimorphism (yeast cells and hyphal cells) is known to be a virulence property of the pathogen C. albicans (Felk et al., 2002). The ability of C. albicans to transform into hyphae has been considered a pathogenic determinant in the initial processes of superficial tissue invasion (Naglik et al., 2003). Hyphae may promote the adherence and penetration of C. albicans to host tissue. SAP 1, SAP 2 and SAP 3 are predominantly expressed in yeast cells, however, SAP 4, SAP 5, and SAP 6 are hyphae-specific genes (Chen et al., 2002; Hube et al., 1994; Lee et al., 2009). In animals, SAP 4-6 isoenzymes are important for the normal progression of systemic infection (Borg-von Zepelin et al., 1999). By promoting the proteolytic degradation of E-cadherin in epithelial adherences junctions, C. albicans can invade mucosal tissues. Recent research shows that SAP 5 is responsible for E-cadherin degradation in vitro (Villar et al., 2007). SAP 5 and SAP 6 may facilitate the penetration of C. albicans hyphae through the epithelium and extracellular matrix. The role of SAP 6 involved in the pathogenesis of C. albicans keratitis is associated with the morphogenic transformation of C. albicans yeasts into invasive filamentous forms (Hua et al., 2009; Jackson et al., 2007; Moran et al., 2004). SAP 4-6 have optimally active at pH 5.0. It implicates that SAP 4-6 could also act as cytolysins in microphages where they are expressed after phagocytosis of the yeast cells (Borg-von Zepelin et al., 1999). SAP 4 to SAP 6 play a significant role in evading host immune defenses. Although Candida dubliniensis is very closely phylogenetically related to C. albicans, it is less frequently associated with human disease and is apparently less virulent than C. albicans. Sequence comparisons revealed that orthologues of SAP 5 and SAP 6 are missing in the C. dubliniensis genome (Loaiza-Loeza et al., 2009).

### III. PEPTIDES

Peptides are comprised of amino acids and are generally less than about 50 residues in length. Examples may include contiguous residues of the SAPs or integrins of 3, 4, 5, 6, 7, 8, 9, 10, 11, 12, 13, 14, 15, 16, 17, 18, 19, 20, 21, 22, 23, 24, 25, 30, 35, 40, 45, 50 or more amino acids in length. Such peptides may be linked to other molecules, for example, by terminal peptide bonds or by other means, as discussed further below. These peptides are believed to be useful in blocking the interaction of SAPs with integrins and thus preventing fungal attack on host cells, leading to fungal dissemination.

A. Synthesis and Purification

Because of their relatively small size, the peptides of the invention can be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

B. Structure

In particular embodiments, peptides will have the general structure:

P₂-P₁-*-P_{1′}-P_{2′}

wherein P₁, P₂ and P_{1′} can be any residue, and P_{2′} is a negatively-charged residue, and -*- indicates modification of the peptide bond into a transition state analog. The peptide is 4-25 residues in length. Negatively-charged residue can be, for example, aspartic acid, glutamic acid, phosphoric acid or sulfonic acid. The peptide may further comprise an XGY motif, wherein X is a positively-charged residue, and Y is a negatively-charged residue. Particular peptides include:

SHLP*S(E/D)FT
RGD-SHLP*S(E/D)FT
SHLP*S(E/D)FT-RGD.

C. Linked Peptides

The peptide may be linked to other agents, such as an RGD-containing protein, such as Integrilin®. Alternatively, the peptide may be is linked to a drug, such as an anti-fungal agent (discussed below in Section V) or a transition state inhibitor.

Crosslinkers suitable for use in accordance with these peptides are well known to those of skill in the art. Table 1 illustrates several crosslinkers.

TABLE 1
HETERO-BIFUNCTIONAL CROSS-LINKERS

| | | | Spacer Arm | |
|---|---|---|---|---|
| | | | Length\after cross- | |
| linker | Reactive Toward | Advantages and Applications | linking | |
| SMPT | Primary amines | Greater stability | 11.2 | A |
| | Sulfhydryls | | | |
| SPDP | Primary amines | Thiolation | 6.8 | A |
| | Sulfhydryls | Cleavable cross-linking | | |
| LC-SPDP | Primary amines | Extended spacer arm | 15.6 | A |
| | Sulfhydryls | | | |
| Sulfo-LC-SPDP | Primary amines | Extended spacer arm | 15.6 | A |
| | Sulfhydryls | Water-soluble | | |
| SMCC | Primary amines | Stable maleimide reactive group | 11.6 | A |
| | Sulfhydryls | Enzyme-antibody conjugation | | |
| | | Hapten-carrier protein | | |
| | | conjugation | | |
| Sulfo-SMCC | Primary amines | Stable maleimide reactive group | 11.6 | A |
| | Sulfhydryls | Water-soluble | | |
| | | Enzyme-antibody conjugation | | |
| MBS | Primary amines | Enzyme-antibody conjugation | 9.9 | A |
| | Sulfhydryls | Hapten-carrier protein | | |
| | | conjugation | | |
| Sulfo-MBS | Primary amines | Water-soluble | 9.9 | A |
| | Sulfhydryls | | | |
| SIAB | Primary amines | Enzyme-antibody conjugation | 10.6 | A |
| | Sulfhydryls | | | |
| Sulfo-SIAB | Primary amines | Water-soluble | 10.6 | A |
| | Sulfhydryls | | | |
| SMPB | Primary amines | Extended spacer arm | 14.5 | A |
| | Sulfhydryls | Enzyme-antibody conjugation | | |
| Sulfo-SMPB | Primary amines | Extended spacer arm | 14.5 | A |
| | Sulfhydryls | Water-soluble | | |
| EDC/Sulfo-NHS | Primary amines | Hapten-Carrier conjugation | 0 | |
| | Carboxyl groups | | | |
| ABH | Carbohydrates | Reacts with sugar groups | 11.9 | A |
| | Nonselective | | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### IV. ANTIBODIES AND PREPARATION THEREOF

In another aspect, the present invention contemplates an antibody that is (a) immunoreactive with a SAP RGD motif, (b) immunoreactive with a SAP integrin binding site, or (c) an anti-idiotype of (a), which would act in the same fashion as (b). An antibody can be a polyclonal or a monoclonal antibody. Antibodies can be whole, single chain, scFV, or fragments (e.g., F′ ab). They may also be chimeric or humanized. Such antibodies are believed to be useful in blocking the interaction of SAPs with integrins and thus preventing fungal attack on host cells, leading to fungal dissemination.

A. Antibody Production

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide or peptide. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

C. Antibodies Linked to Other Agents

As appropriate, antibodies in accordance with the present invention can be linked to other agents, such as antifungals (discussed below), and may utilize linking technologies described above.

### V. ADDITIONAL ANTIFUNGAL TREATMENTS

Fungal intrinsic and acquired resistance to antibiotics represents a major problem in the clinical management of fungal infections. Thus, the present invention also provides for new multi-drug therapy regimens because, while many fungal infections may be effectively treated by a traditional antifungal agent, other infections may be treated more effectively using one or more additional agents. Such multi-drug combinations may also reduce the amount of drug needed (and hence the side effects ensuing therefrom), or more quickly limit or eliminate the infection.

To kill fungi, inhibit fungal cell growth, or otherwise reverse or reduce the emergence of drug-resistant variants using the methods and compositions of the present invention, one would generally contact a “target” cell with an agent (peptide, antibody) according to the present invention and another antifungal compound. The compositions would be provided in a combined amount effective to kill fungi or inhibit fungal cell growth. This process may involve contacting the cells with the agents at the same time. This may be achieved by contacting the cell with a single composition or pharmacological formulation that includes both agents, or by contacting the cell with two distinct compositions or formulations at the same time.

The treatment according to the present invention may precede or follow the other agent by intervals ranging from minutes to hours to days. In embodiments where the agent according to the present invention and the other agent are administered separately, one would generally ensure that a significant period of time did not expire between the time of each delivery, such that the agent according to the present invention and the other agent would still be able to exert an advantageously combined effect on abrogating the fungal infection. In such instances, it is contemplated that one would administer both modalities within about 12-24 hours of each other and, more particularly, within about 6-12 hours of each other, including 1, 2, 3, 4, 5, 6, 7, 8, 12, or 24 hours. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Equally it may be necessary to administer multiple doses of the agent according to the present invention and/or the other agent in order to achieve the desired effectiveness. Various combinations may be employed, where the agent according to the present invention is “A” and the other agent is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve fungal cell killing, both agents are delivered to a cell in a combined amount effective to kill the cell and remove the infection.

Traditional antifungal treatments that are suitable for use in combination with the present invention include polyenes, amphotericin B, filipin, nystatin, allylamines (terbinafine and naftifine), echinocandins (caspofungin or MK-0991, V-echinocandin, FK643), sordarins, azosordarins, flucytosine and griseofulvin. Other agents include the imidazoles and the N-substituted triazoles. While more of the former are currently in use, more recent efforts have focused on the triazoles given their more slow metabolism and the lesser effect on human sterol synthesis. Currently used imidazoles include chlormidazole, clotrimazole, miconazole, isoconazole, ketoconazole, econazole, bifonazole, butoconazole, democonazole, fenticonazole, lanoconazole, lombazole, oxiconazole, sertaconazole, sulconazole and tioconazole, UR-9746, UR-9751 vibunazole. Fluconazole, terconazole, genaconazole, itraconazole, voriconazole, posaconazole, ravuconazole, parconazole, T-8581 (Yotsuji et al., 1997), BMS 207147 (Fung-Tomc et al., 1999), SS 750 (Takeda et al., 2000), TAK 456, TAK 457, R-102557 (Oida et al., 2000), UR-9751, R-120758 (Kamai et al., 2000), and SYN 2869 (Johnson et al., 1999).

Fluconazole: Fluconazole is a fluorinated bis-triazole. It is almost completely absorbed from the GI tract. Concentrations in plasma are essentially the same when the drug is given orally or intravenously, and bioavailability is not altered by food or gastric activity. Human adult dosages are in the range of 50 to 400 mg daily, with both oral and intravenous formulations available.

Ketoconazole: Ketoconazole is administered orally and is used to treat a number of superficial and systemic fungal infections. Oral absorption varies between individuals. Simultaneous administration of H₂ histaminergic receptor blocking agents and antacids may limit bioavailability. Oral doses range from 200-800 mg, giving peak plasma concentrations of 4-20 μg/ml.

Miconazole: Miconazole is a close relative of econazole. It readily penetrates the strateum corneum and persists for more than 4 days after application. Less than 1% is absorbed from the blood. It is available as a 2% dermatologic cream, spray, powder or lotion, 100 and 200 mg suppositories (7 day or 3 day regimen, respectively).

Itraconazole: Itraconazole is a triazole closely related to ketoconazole. Absorption in the fasting state is 30% of that when the drug is take with food. Although concentrations of this drug in plasma are much lower than with the same doses of ketoconazole, tissue concentrations are high. Concurrent administration of rifampin decreases concentrations of itraconazole in plasma substantially. Typical oral dose for adults is 200 mg once daily, but higher doses may be used for limited duration.

Clotrimazole: Clotrimazole is a topical antifungal. Absorption is less than 0.5% after application to the skin, but 3-10% from the vagina. Typical dosage is as a 1% cream lotion or solution. It also is used in 100 or 500 mg vaginal tablets and 10 mg troches. Skin applications are twice a day; vaginal regimens include one 100 mg tablet per day for 7 days, the 500 mg tablet used once, or 5 g cream for 7-14 days.

Econazole: Econazole is the deschloro derivative of miconazole. It readily the penetrates the stratum corneum and is found in effective concentrations down to the mid-dermis. Less than 1% appears to be absorbed into the blood. It is provided in a 1% cream applied twice per day.

Terconazole: Terconazole is a ketal triazole with structural similarity to ketoconazole. It is available as an 80 mg suppository inserted vaginally at bedtime for three days, or as a 0.4% vaginal cream used for 7 days.

Butoconazole: Butoconazole is comparable to clotrimazole and is available as a 2% vaginal cream. Typical treatment regimen is once a day application for three days.

Oxiconazole: Oxiconazole is a topical antifungal for treatment of common pathogenic dermatophytes. It is available in a 1% cream.

Sulconazole: Sulconazole is a topical antifungal for treatment of common pathogenic dermatophytes. It is available in a 1% solution.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA Office of Biologics standards.

### VI. PHARMACEUTICAL FORMULATIONS AND ROUTES OF ADMINISTRATION

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The agents of the present invention will often be formulated for parenteral administration, e.g., formulated for injection via the intravenous, intramuscular, sub-cutaneous or other such routes, including direct instillation into an infected or diseased site. The preparation of an aqueous composition that contains an azole potentiator agent as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection also can be prepared; and the preparations also can be emulsified.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

The compositions can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. Formulations are easily administered in a variety of dosage forms, such as the type of injectable solutions described above, but drug release capsules and the like also can be employed.

Suitable pharmaceutical compositions in accordance with the invention will generally include an amount of the composition admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th ed., Mack Publishing Company, 1980, incorporated herein by reference. It should be appreciated that, for human administration, preparations should meet sterility, pyrogenicity, general safety and purity standards as required by the FDA Office of Biological Standards.

The therapeutically effective doses are readily determinable using an animal model, as shown in the studies detailed herein, or by comparing the agents with known antifungal drugs. Experimental animals bearing bacterial or fungal infection are frequently used to optimize appropriate therapeutic doses prior to translating to a clinical environment. Such models are known to be very reliable in predicting effective anti-bacterial and antifungal strategies.

In addition to the compounds formulated for parenteral administration, such as intravenous or intramuscular injection, other pharmaceutically acceptable forms also are contemplated, e.g., tablets or other solids for oral administration, time release capsules, liposomal forms and the like. Other pharmaceutical formulations may also be used, dependent on the condition to be treated.

For oral administration, the agents of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The inventors propose that the local or regional delivery of the agents according to the present invention will be a very efficient method for delivering a therapeutically effective composition to counteract the clinical disease. Alternatively, systemic delivery of may be the most appropriate method of achieving therapeutic benefit from the compositions of the present invention.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Arm A of C. albicans SAPs 4, 5 and 6 Contains Integrin Binding Motifs

The inventors compared the amino acid sequences of porcine pepsin with C. albicans SAPs 4, 5 and 6 by homology alignment and found that the “Arm A” in these SAPs is achieved largely by insertions of about 7 amino acids between residues 42 and 50 of pepsin (FIG. 1B). In addition, they found that these three SAPs contain a known integrin binding motif, a single RGD motif in SAPs 4 and 5, and two RGD motifs in SAP 6 (FIG. 1B). A less effective integrin binding KGD motif is also found in SAP 5. Since integrin is a cell surface adhesion protein, the inventors postulated that SAPs 4-6 can bind cells via RGD-integrin binding and such interaction may function in the virulence of C. albicans infection. They designed experiments to demonstrate the binding of these three SAPs to cells via by interaction with integrin.

RGD motif in C. albicans SAP 4-6 Subfamily. The set of structures of isoenzyme subfamily SAP 1-3 has been resolved (Abad-Zapatero et al., 1996; Borelli et al., 2007; Cutfield et al., 1995). SAP 5, in complex with pepstatin A at 2.5 Å resolution, has been described recently (Borelli et al., 2008). This is the first three-dimensional structure of subfamily SAP 4-6 member. Structural analysis reveals a highly conserved overall secondary structure of SAP 1-3 and SAP 5. An in silico analysis was performed of the C. albicans SAP 4-6 isoenzyme subfamily. Sequence alignment reveals a highly conserved integrin-binding motif RGD close to the C-terminus (FIG. 1). However, there appears to be no RGD motif present in the other SAP subgroups of C. albicans after examination of the gene contexts. This significantly implies that SAP 4 to SAP 6 can interact with the adherent receptors on cellular surface of the host.

The crystal structures of SAPs display a crab-shaped architecture. The flap loops at the entrance to the active site cleft is similar to the powerful claws of the crab. These claws are similar to Arms, which may functionally catch the potential targets. The “RGD” motif of SAP 4-6 is located at Arm I region (FIG. 2). Interestingly, there are even two continuation integrin-binding motifs RGD on the Arm I of SAP 6. This may imply a much stronger adherent role of SAP 6 during the process of C. albicans infection of the host. Interestingly, there are two motifs “YYT” and “DXXG” are located at the other two arms respectively. The DXXG motif can functionally bind with Mg²⁺ in GTPase superfamily. The protein tyrosine (Y) phosphatase is related Caspase-3 regulated apoptotic cell death (Halle et al., 2007; Rafiq et al., 2006).

### Example 2

### Demonstration of SAP 6 Binding to Integrin on Cell Surface, then Enter the Cell and Cause Cell Death

Cellular integrin binds SAPs. As discussed above, based on the structural and functional analysis, the inventors identified an integrin-recognition motif (RGD) highly conserved in SAP 4-6 subfamily of C. albicans. The enzymes of this subfamily have an optimum pH near 5.0. It implies that SAP 4 to 6 might play a critical role on the pathogen-host cell interaction during the initial process of the adhesion and subsequent C. albicans infection, for instance, endocytic pathway in live cells and eventually apoptosis.

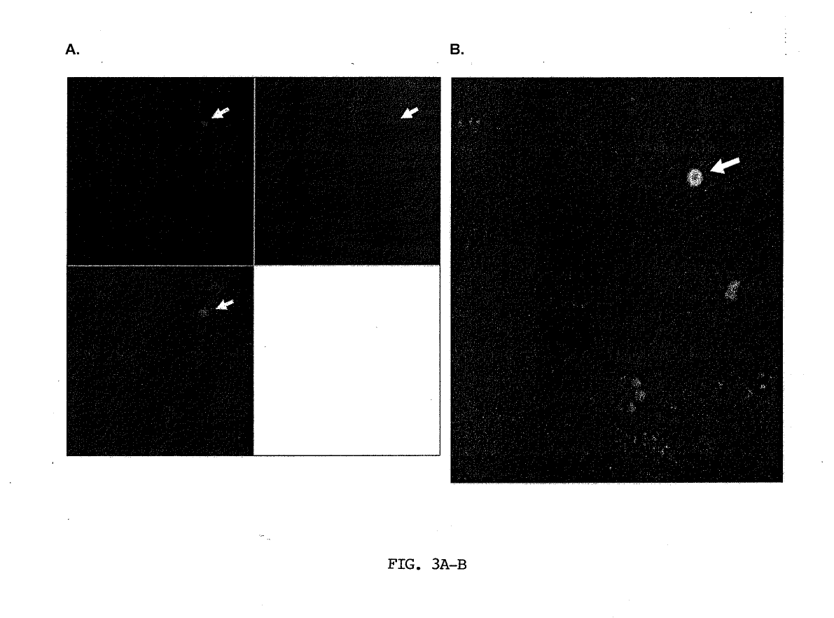

SAP 6 binds to Human Platelets. Recombinant SAP 6 was expressed in the yeast Pichia according to Borg-von Zapelin et al. (1998) and purified (unpublished results, Wu and Tang). Recombinnat C. albicans SAP 6 was labeled with Alexa Fluor® 488 based on the Invitrogen Alexa Fluor® 488 protein labeling kit manual. Crude human platelets were obtained from Oklahoma Blood Institute. To obtain the pure platelets, it needs to be further isolated as follows: (1) carefully transfer 10 ml platelets (including rich plasma) to a 15 ml tube and add 1/10 volume of ACD anticoagulant (6.25 g sodium citrate.2H₂O, 3.1 g citric acid anhidrous, 3.4 g D-glucose in 250 ml H₂O); (2) pellet platelets by centrifuged at 3000 rpm for 5 minutes at room temperature (note: after centrifugation, supernatant still contains significant amount of platelets and it can be collected for experiments); (3) resuspend the pellet in ˜1 ml Hepes-Tyrode buffer pH=7.4 (134 mM sodium chloride, 12 mM sodium bicarbonate, 2.9 mM potassium chloride, 0.34 mM sodium phosphate monobasic, 5 mM Hepes, 5 mM glucose, 1% BSA). When the human platelets were ready, they were incubated labeled SAP 6 with relative amount of platelets at 4° C., room temperature and 37° C. for 2 h, 1 h and 30 min respectively. The mixtures were washed by Hepes-Tyrode buffer two times at 1500 rpm for 8 min. Resuspended the pellets in Hepes-Tyrode buffer and mounted on glass slides. The glass slides were imaged by the epifluorescence microscope for simultaneous detection of Platelet/SAP 6-AlexaFluor-488 fluorescence (FIGS. 3A-B).

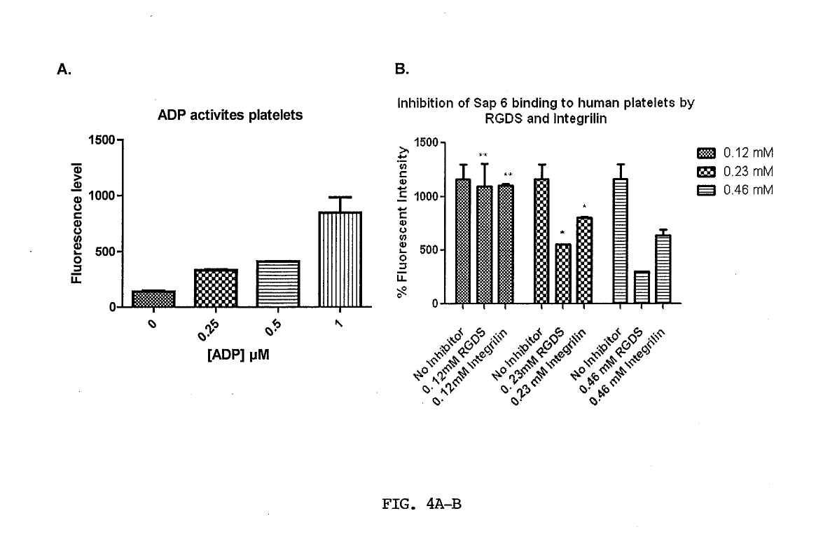

SAP 6 binds to human platelet competed by the RGDS peptide and Integrilin® drug. The inventors determined that SAP 6 binds to human platelet through specific motif by the peptide drugs. The RGDS peptide (Arg-Gly-Asp-Ser), Fibronectin (1 mg/ml solution) and ADP are from Sigma®. Integrilin® (C₃₅H₄₉N₁₁O₉S₂ and molecular weight is 831.96) was obtained from Schering Corporation Kenilworth, N.J. 07033 USA. Purified recombination C. albicans SAP 6 was labeled by the Alexa Fluor° 488 Protein Labeling Kit from Invitrogen. The fresh human platelet was obtained from Oklahoma Blood Institute. Fifty μl purified human platelets were added into the total 100 μl reaction mixture. A gradient of ADP concentrations (0.25 μM, 0.5 μM and 1 μM) was designed to test if ADP can activate platelets during the binding assay (FIG. 4A). Dose-dependent inhibition assay of RGDS and Integrilin® was performed by measuring the fluorescence of TECAN (Ex./Em.=488 nm/519 nm) (FIG. 4B).

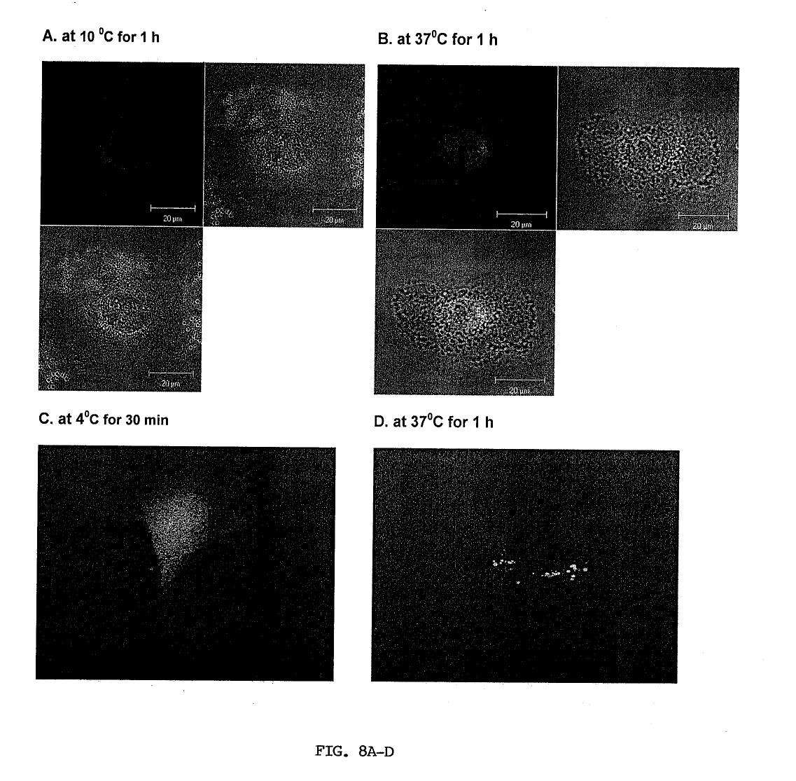

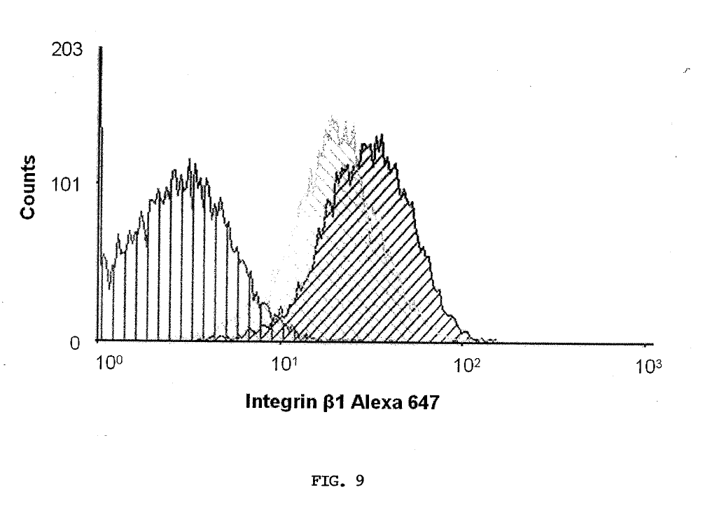

SAP 6 binds to human lung carcinoma cell A549 and endocytosis assay at 37° C. Since the inventors demonstrated that SAP 6 can bind to human platelet through the specific intergin-binding motif RGD. Then, the other hypothesis immediately needs to be figure out whether SAP 4-6 subfamily exist the endocytic pathway in live cells and eventually trigger the caspase-3 regulated apoptosis. To prove this hypothesis, the inventors changed the cell line to perform further cellular experiments. The inventors compared the binding assays of SAP 6 with human lung carcinoma cells between 10° C. and 37° C. by fluorescent confocal microscopy. Data show that at 10° C. for 30 min, SAP 6 mainly on the cell surface (initial binding), however, at 37° C. for 60 min, there are lots of strongly green signal dots, which significantly indicate that endocytosis happened in A 549 cells (FIGS. 8A-D).

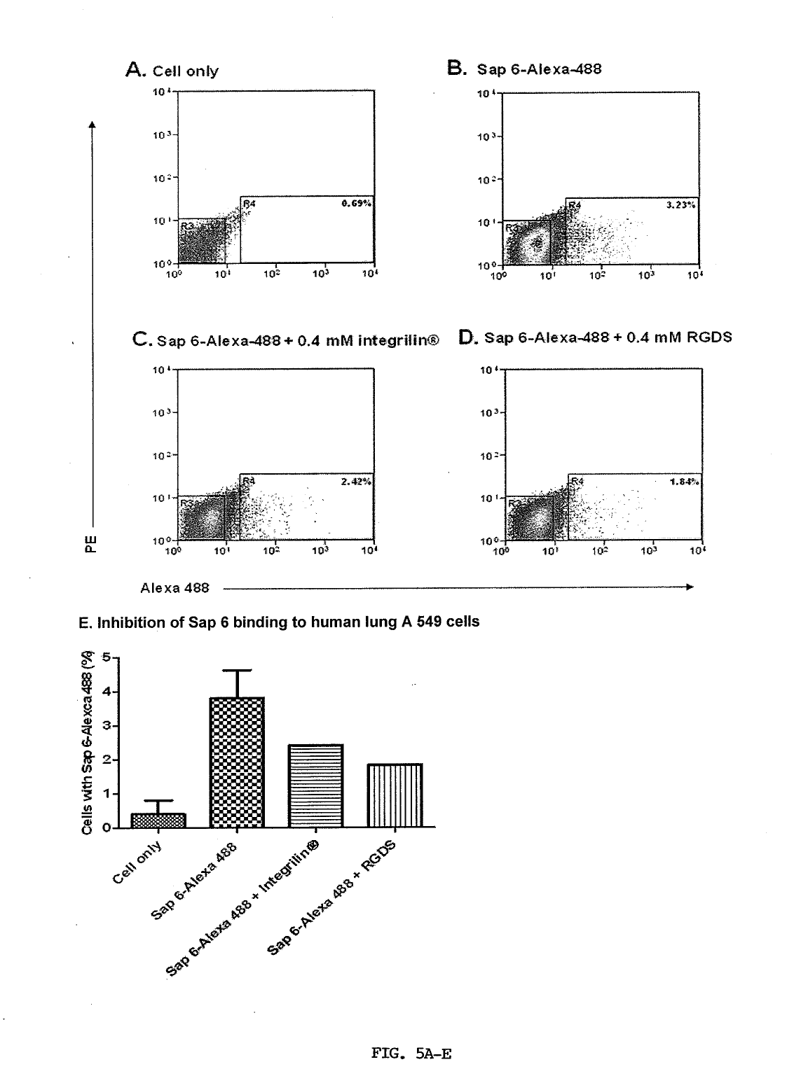

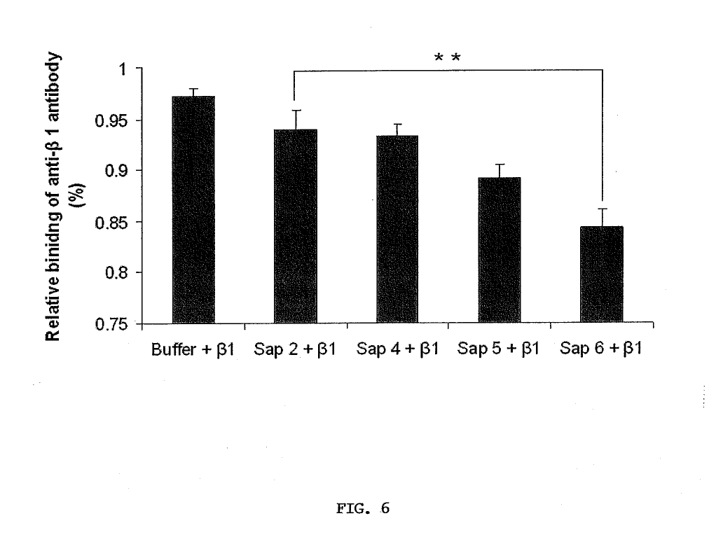

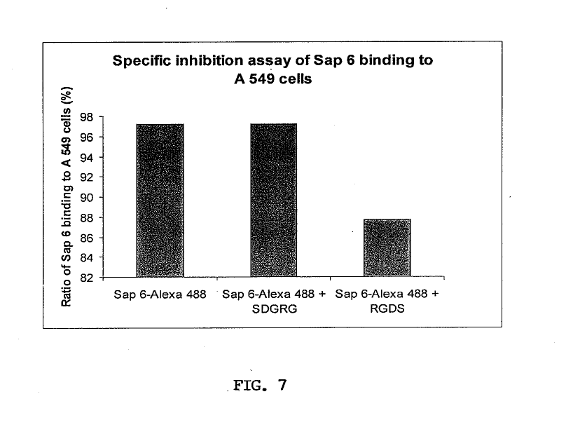

RGDS peptide or Integrilin® inhibition assay and cell viability assay. Grew human lung carcinoma A 549 cell and harvested the cells with 46×10⁴ per ml. Changed the medium from complete growth medium into D-MEM/F-12 without phenol red; and transferred the aliquot volume of 0.2 ml cells into 1.5 ml sterile tubes. Added 50 μl of 2.4 mM RGDS peptide and Integrilin® into sample III and sample IV respectively, incubated all samples at 37° C. for 10 min. Then, added 50 μA labeled Fluoro®-488 SAP 6 into Sample II, Sample III and Sample IV (Sample I is negative control, added 100 μl medium inside). Incubated the four mixtures at 10° C. for 30 min, and then centrifuged the cell cultures at 400 g at 10° C. for 5 min to remove the suspension. Washed the cells 1-2 times with 1 ml D-MEM/F-12 medium without phenol red to remove the unbound compounds. Sort the negative (non-labeling SAP 6) and positive (binding labeled SAP 6) cells by fluorescence-activated cell-sorting (FACS). RGDS peptide and Integrilin® can inhibit SAP 6 initially bind to human lung carcinoma cell (A549) significantly; RGDS has much more inhibition than that of Integrilin®, which is the same as platelet cellular experiment. RGDS is near half inhibition (1.68%) compared with the positive control (3.23%) (FIGS. 5A-E). To further prove that SAP 6 bind to A 549 through the specific RGD motif, the inventors compared the inhibition of RGDS and SDGRG. SDGRG is the reverse peptide of SRGD, which has no competition to bind to integrin with RGDS. It is often considered as a negative control. Here, the inventors found that SDGRG is significantly less inhibition for SAP 6 to bind to A549 cells (FIG. 7).

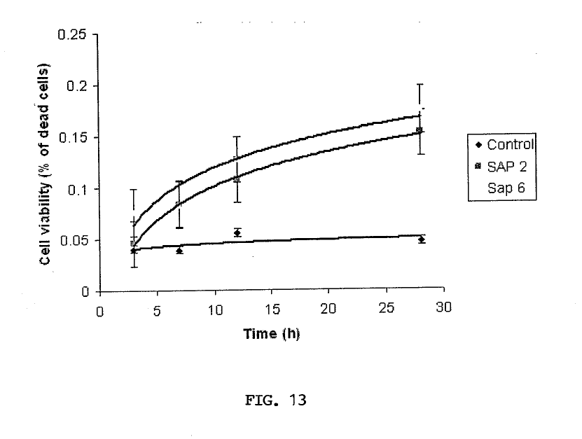

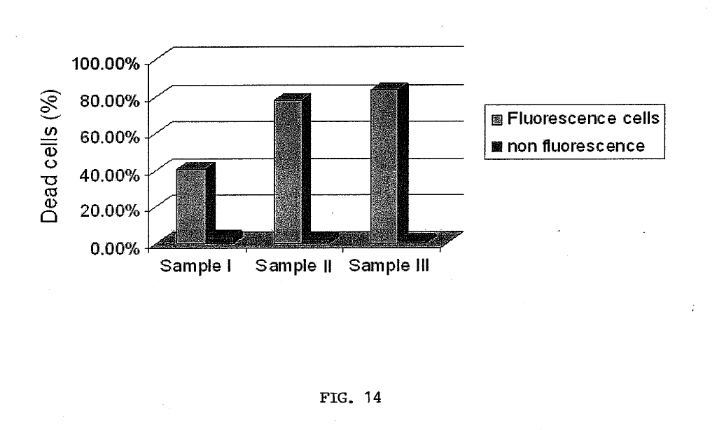

Apoptosis assay of SAPS 2 and 6 by Trypan Blue. To detect the apoptosis of the negative and positive cells in FIG. 7; the inventors continued to incubate the fluorescence labeled cells and non-fluorescence labeled cells at 37° C. for 4 h. Counted by Trypan Blue immediately after cell sorting by FACS, there were almost no dead cells in either the fluorescence labeled cells or non-fluorescence labeled cells. However, after incubated at 37° C. for 4 h, the stained cells (mainly apoptosis & necrosis) of fluorescence labeled cells (positive cells) are much more than that of and non-fluorescence labeled cells (negative cells) (FIGS. 13-14).

Apoptosis assay of SAPs from C. albicans. To determine if other C. albicans SAPs can induce apoptosis, the inventors used an epithelial cell line of human lung carcinoma A549 to perform apoptosis experiment by flow cytometry. SAP 2 and SAPs 4-6 were each used in the same buffer (10 mM HEPES, pH 7.0, 150 mM NaCl) with 1 μM final concentration. HEPES buffer and 10 μM Camptotchecin (Sigma) was used as a negative- and positive-induced control respectively. After seeding of 200 μl A549 cells into 48-well cell culture plate at 54×10⁴ cells per ml, the inventors added the SAPs and control samples into the same cell culture plate, continually incubated the cell cultures at 37° C., and harvested the cells by using 1× Cell Dissociation Solution without enzyme (Sigma) after 11 hrs incubation. The cells were washed once with cool 1×PBS, resuspended the cells in 100 μl 1× apoptosis binding buffer, and 5 μl 7AAD and 5 μl Annexin-PE V were added into the relative cultures. After incubating the mixtures in dark for 15 min at room temperature, another 400 μl 1× apoptosis binding buffer was added to the cultures in 5 ml tubes, and Flow Cytometry (BD FACSCalibur™) was performed.

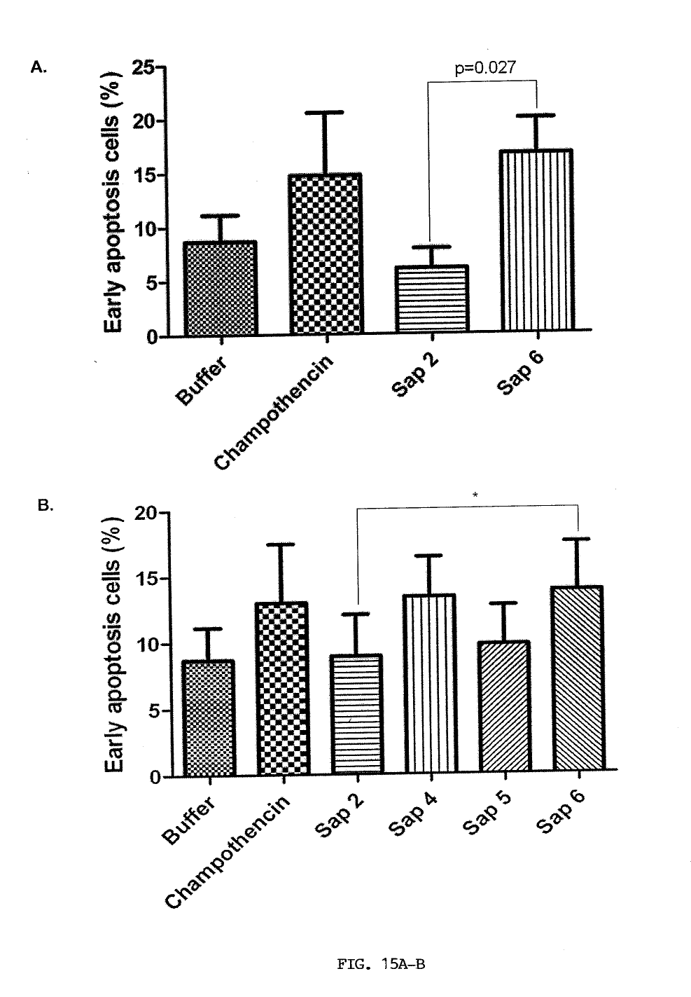

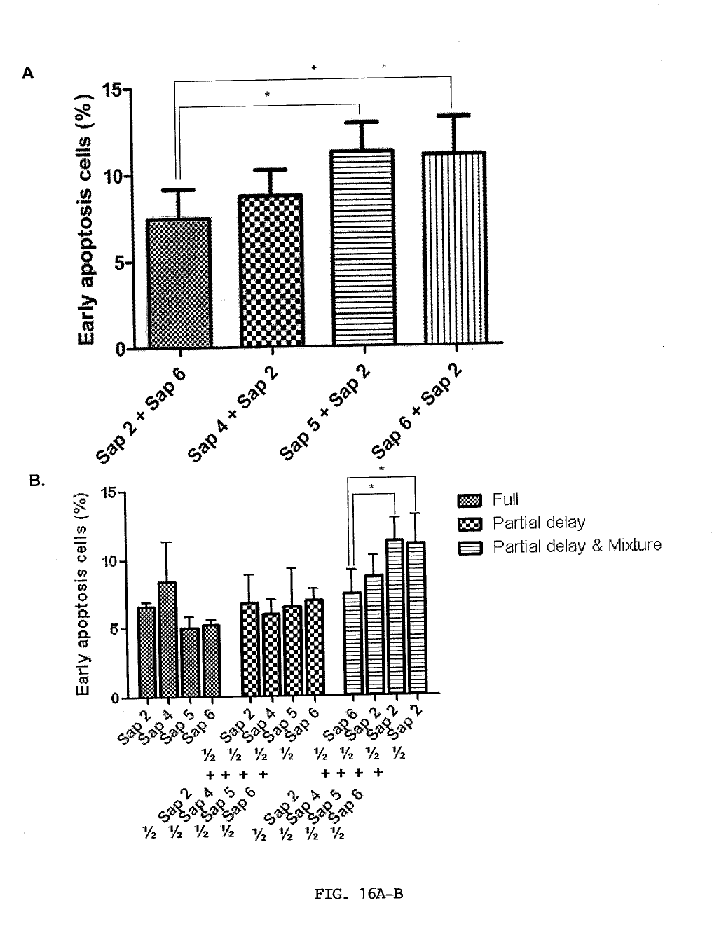

Within 12 hrs of incubation at 37° C., SAP 2, SAP 4-6 induced apoptosis, alone and in combination (FIGS. 15-16). The data significantly agrees with the hypothesis that SAPs can initially adhere to epithelial cells and following trigger the apoptosis. During C. albicans parthenogenesis, the SAP 4-6 subfamily performs the critical pioneer role for the SAP 1-3 subfamily.

One interesting observation comes from FIG. 16, which indicates that when the inventors combined the enzymes to treat the A549 cells, and at the different time points added different enzymes, the model of “SAP 6+SAP 2” induced much more early apoptosis or LMP compared with other combination models. So, it is clear that the SAP 4-6 subfamily can perform the critical pioneer role for the SAP 1-3 subfamily. SAP 4-6 can initially bind to the cell surface receptor of epithelium cells through integrin-binding motif RGD, and thereafter further induce endocytosis at 37° C. Once SAP 4-6 traffics from the early endosome to lysosome, it can induce LMP of the cells. After lysosomal membrane permabilization, it may trigger the caspase-3 regulated apoptosis in cytosol. When the hosts become weak from apoptosis (which is triggered by the SAP 4-6 subfamily), the SAP 1-3 subfamily (especially SAP 2) can quickly spread to deep organs concomitantly with tissue destruction. Therefore, during C. albicans parthenogenesis, SAP 4-6 subfamily performs the critical pioneer role for SAP 1-3 subfamily; and the SAP 1-3 subfamily (especially SAP 2) allow C. albicans to thrive within the destroyed tissue by degrading host proteins for nutrient supply. Thus, the SAP 4-6 subfamily is critically important for drug targeting in the development of a new antifungal drug against Candida infection.

### Example 3

### Demonstration of Subsite Specificity of Candida albicans SAP 4, SAP 5 and SAP 6

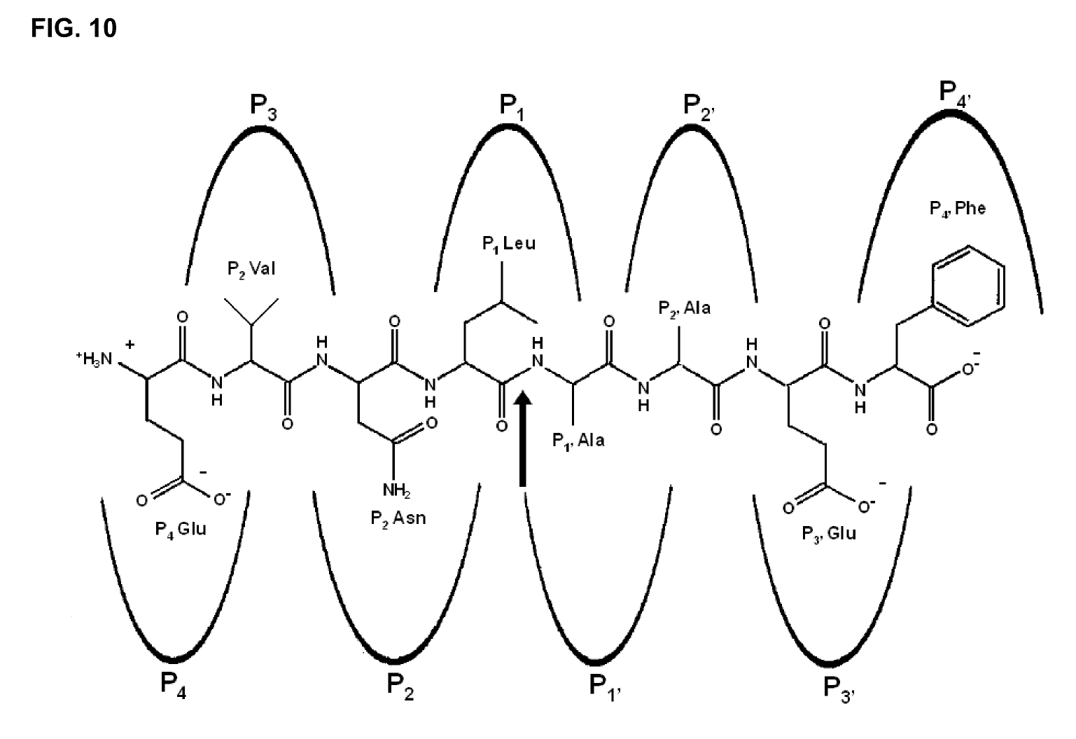

Subsite specificity of aspartic proteases, including C. albicans SAPs, are important for the design of inhibitors. Most aspartic proteases can bind 8 substrate residues in their active site cleft. The subsites in the substrates of proteases are by convention, named as in FIG. 10. For example, the inventors determined the preliminary subsite specificity of memapsin 2 (Lin et al., 2000) which led to the design of potent inhibitors (Ghosh et al., 2000).

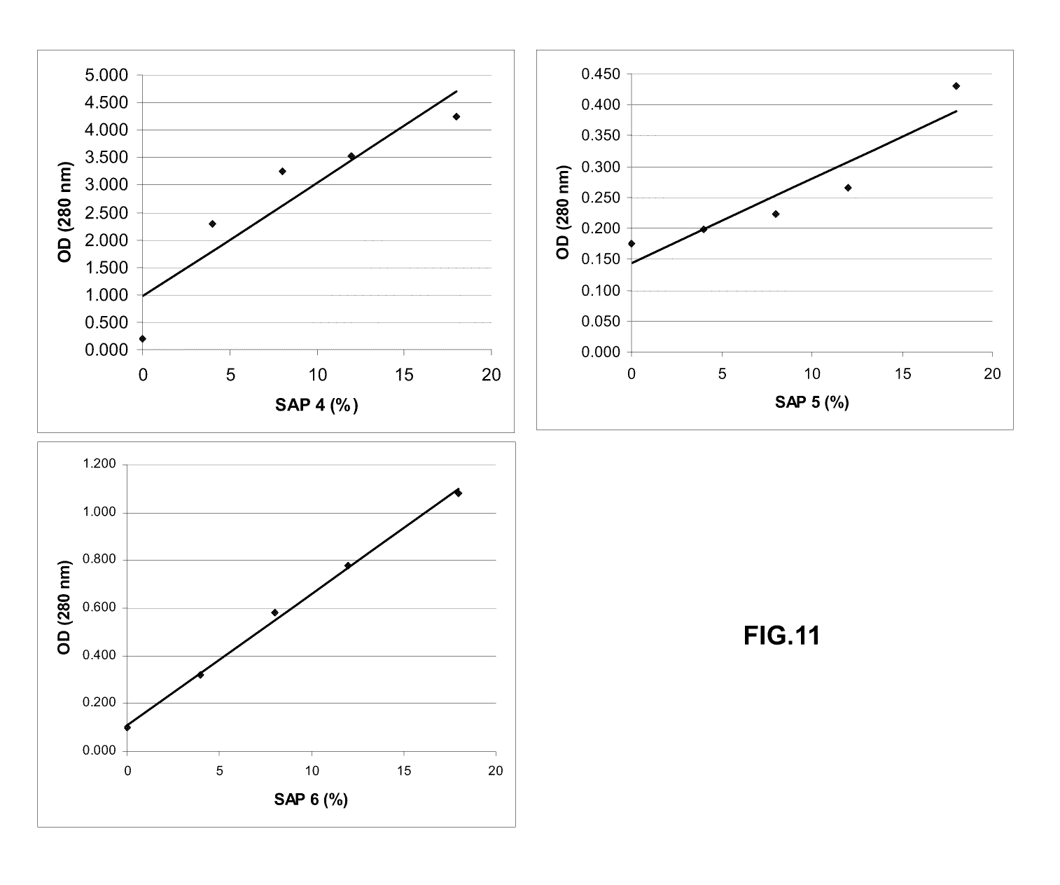

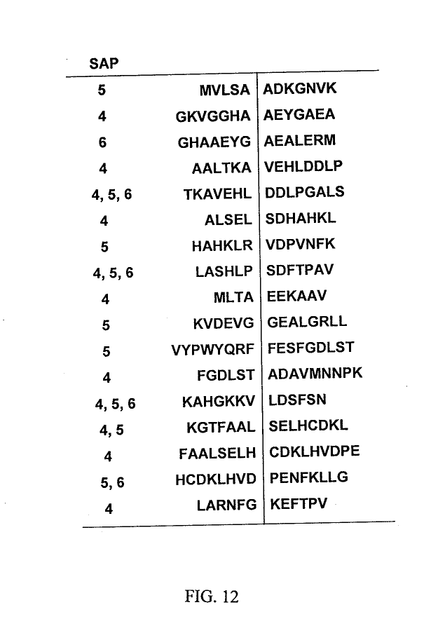

In order to determine subsite specificity of C. albicans SAPs 4-6, the inventors incubated the purified proteases separately with globin chains (mixture of α and β chains) from bovine hemoglobin and determined that the proteins are hydrolyzed by C. albicans SAPs 4-6 (FIG. 11). They then analyzed the globin peptide fragments resulting from three digestions in MALDI-TOF mass spectrometer. The positions of the peptides in the sequence of globin chains were identified by their mass. With these data, the positions of proteolytic cleavage were identified as shown in FIG. 12 in which the subsites are aligned. A clear preference of P2′ for a negatively-charged residue, either aspartic acid (D) or glutamic acid (E), was found for SAP 5-7, as can be seen in red letters in FIG. 12. No other clear consensus residue was found in other subsites. Although the inventors did not identify each of the sites in FIG. 12 to be hydrolyzed by all three SAPs, the inventors expect that theses sites will all be cleaved by three SAPs. It is possible, however, that three SAPs may have somewhat different rates for the site. These results indicated that these three SAPs have a major specificity preference of a P2′ negatively-charged residues.

### Example 4

### Data Showing SAP Intracellular Trafficking and Effects on Apoptosis

Immunofluorescence colocalization of SAP 6 with early endosome and lysosome occurs at different times. SAP 6 colocalizes with early endosome and lysosome. In data not shown, SAP 6-Alexa Fluoro® 488 was incubated with the early endosome marker EEA1 for 9 hrs at 37° C., or with lysosome marker LAMP1 for 15 hrs at 37° C. Results show that SAP 6 colocalized with early endosome and lysosome respectively at different time points incubated at 37° C.

Immunofluorescence colocalization of SAP 6 with integrin P1 on the cell surface of A549. In data not shown, immunofluorescence colocalization of SAP 6 with integrin β1 on the cell surface of A549 was assessed. A549 cells were seeded (7.2×10⁴ cells) in 8-well Lab-Tek® II chambers, 80 μA of 16.6 μM SAP 6-Alexa Fluoro® 488 was added into A549 cells and incubated at 100° C. for 1 h; then 6 μl mouse anti-human integrin 131 monoclonal antibodies were added, which recognize the extracellular domain of integrin, followed by incubation for 20 min at 37° C. to allow for binding. After being rinsed with cold 1×PBS three times, the cells were fixed with 4% paraformaldehyde (Wt/v in PBS) on ice for 15 min. After rinsing the cells, 10 μl donkey anti-mouse IgG directly conjugated Cy3 antibody was added and incubated at RT for 3 h. Cells were rinsed three times with cold 1×PBS, then Visualized by Zeiss LSM510 confocal. Patterns show that SAP 6 colocalized with integrin β1 on the cell surface of A549.

Immunofluorescence colocalization of internalized intergin β1 with SAP 6 inside A549 cells. In data not shown, immunofluorescence colocalization of internalization of SAP 6 with integrin inside A549 cells was assessed. A549 cells were seeded (7.2×10⁴ cells) in 8-well Lab-Tek® II chambers, 80 μl 16.6 μM SAP 6-Alexa Fluoro® 488 was added into A549 cell culture and incubated at 10° C. for 2 h; 6 μl mouse anti-human integrin β1 monoclonal antibodies was then added, which recognizes the extracellular domain of integrin, for 1 h at 37° C. to allow for internalization of integrin and SAP 6. After being rinsed with cold 1×PBS three times, the cells were fixed with 4% paraformaldehyde (Wt/v in PBS) at RT for 15 min. Cells were incubated with an excess amount of unconjugated anti-mouse IgG (3 μl, 25.2 mg/ml) to block the antibody remaining on the cell surface. The cells were permeabilized with 0.2% Saponin for 15 min at RT. After rinsing the cells three times with cold 1×PBS, 10 μl donkey anti-mouse IgG directly conjugated Cy3 antibody was added and incubated at 37° C. for 1 h. The cells were rinsed three times with cold 1×PBS, and visualized by Zeiss LSM510 confocal. The patterns show that SAP 6 together with integrin were internalized and colocalized inside A549 cells.

Real-time fluorescence imaging shows that SAP 6 can be endocytosed in A549 cell at 37° C. In data not shown, the real-time fluorescence imaging showed that SAP 6 was endocytosed into A549 cell after incubation at 37° C. for 1 h.

Morphology assay shows that RGDS peptide can rescue the apoptosis of A549 cells induce by SAP 2 and SAP 4-6. In data not shown, morphology of bright-field microscope images (10×) of SAP 6 after two weeks incubation showed that RGDS can rescue the apoptosis of A549 cells induced by SAP 6. In additional data not shown, morphology of bright-field microscope images (10×) of SAP 2 and SAP 4-6 after two weeks incubation show that can significantly induce the apoptosis and death of A549 cells (especially SAP 6); however, SAP 2 does not play this role at the early infection stage.

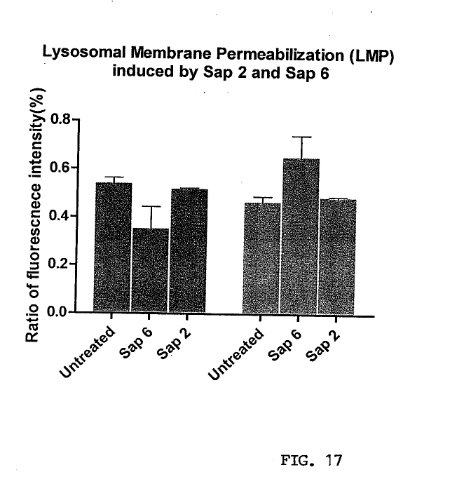

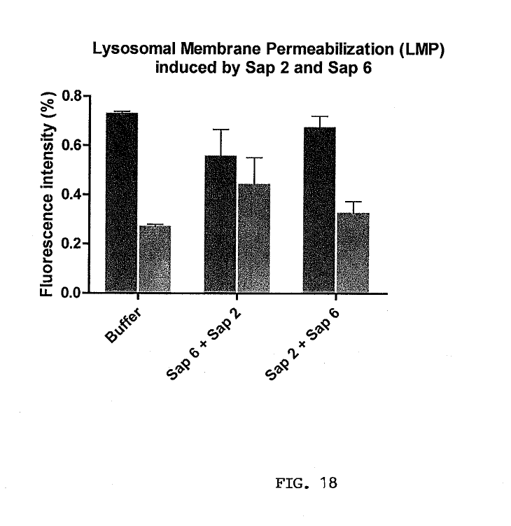

LMP induced by SAP 2 and/or SAP 6. As shown in FIG. 17, lysosomal membrane permeabilization (LMP) was induced by SAP 6, but not by SAP 2. In data not shown, buffer (untreated as a control) showed a red punctate pattern on cells. In A549 cells treated with SAP 6, however, the red punctate pattern was lost, and green fluorescence increased significantly. In A549 cells treated by SAP 2, the red and green fluorescence were not changed significantly. FIG. 18 shows LMP induced by combination of SAP 2 and SAP 6. Similar to the data shown in FIG. 17, here the control showed a red punctate pattern, whereas in A549 cells treated with SAP 6+SAP 2, the red punctate pattern was lost, and the green fluorescence increased significantly. In A549 cells treated by SAP 2+SAP 6, the green and red fluorescence were changed less compared with that of SAP 6+SAP 2.

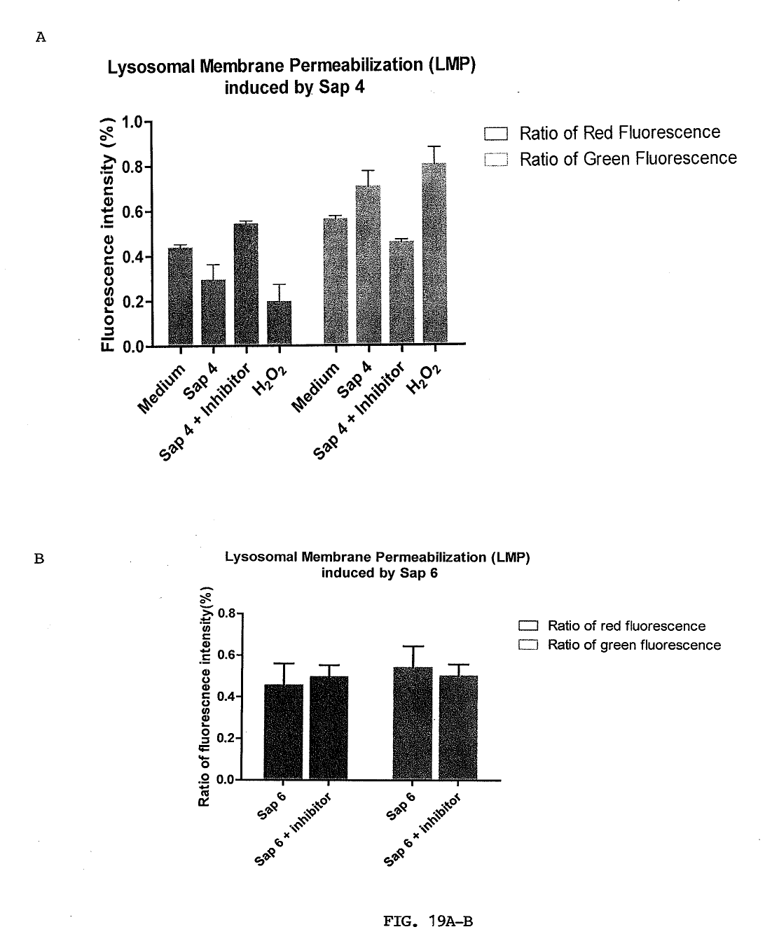

GRL-001-10CAND can inhibit LMP of A549 induced by SAP 4. The synthetic inhibitor of GRL-001-10CAND has an IC50 of ˜193 nM for SAP 4; however, it is not good inhibitor for SAP 5 and SAP 6. In data not shown, a buffer (negative control) gave red punctate pattern (H₂O₂ used as positive control). In A549 cells treated with SAP 4, the red punctate pattern was lost, and green fluorescence increased significantly. A549 cells treated by SAP 4 and GRL-001-10CAND inhibitor, the green and red fluorescence were not changed, meaning that the inhibitor can rescue of LMP of A549 induced by SAP 4. FIG. 19A shows the quantification of the red and green fluorescence intensity. FIG. 19B shows quantification of the red and green fluorescence intensity induced by SAP 6. GRL-001-10CAND cannot inhibit LMP induced by SAP 6 (confocal imaging not shown).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
U.S. Pat. No. 4,196,265
U.S. Pat. No. 4,680,338
U.S. Pat. No. 5,141,648
U.S. Pat. No. 5,563,250
U.S. Pat. No. 5,856,456
U.S. Pat. No. 5,880,270
Abad-Zapatero et al., Protein Sci., 5:640-652, 1996.
Alexander and Perfect, Drugs, 54:657-678, 1997.
Barany and Merrifield, In: The Peptides, Gross and Meienhofer (Eds.), Academic Press, NY, 1-284, 1979.
Blasi et al., Minerva Ginecol, 50:265-76, 1998.
Boerlin et al., J. Clin. Microbiol., 34:1235-1248, 1996.
Borelli et al., Proteins, 68:738-748, 2007.
Borelli et al., Proteins, 72:1308-1319, 2008.
Borg-von Zepelin et al., J. Invest. Dermatol., 113:747-751, 1999.
Borg-von Zepelin et al., Mol. Microbiol. 28:543-554, 1998.
Brajtburg and Bolard, Clin. Micro. Rev., 512-531, 1996.
Cameron et al., Antimicrob Agents Chemother, 37:2449-53, 1993.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Chen et al., Microbiology, 148:3743-3754, 2002.
Clancy et al., Int Arch Allergy Immunol, 118:349-50, 1999.
Cowen et al., J. Bacteriol, 182:1515-1522, 2000.
Cutfield et al., Structure, 3:1261-1271, 1995.
De Brabander et al., Sabouraudia, 18:197-210, 1980.
Dixon et al., Public Health Report., 111:226-235, 1996.
Felk et al., Infect. Immun., 70:3689-3700, 2002.
Fong et al., Genitourin Med., 69:44-6, 1993.
Fong, Clin. Invest. Med., 17:426-31, 1994.
Franz et al., Antimicrob. Agents Chemother, 42:3065-3072, 1998.
Franz et al., Mycoses, 42:453-458, 1999.
Fung-Tomc et al., Diagn. Microbiol. Infect. Dis., 35:163-7, 1999.
Gefter et al., Somatic Cell Genet., 3:231-236, 1977.
Ghosh et al., J. Am. Chem. Soc., 122:3522-3523, 2000.
Goding, In: Monoclonal Antibodies: Principles and Practice, 2d ed., Academic Press, Orlando, Fla., pp 60-61, 71-74, 1986.
Halle et al., Mol. Cell. Biol., 27:1172-1190, 2007.
Henderson and Hirvela, Arch. Urg., 131:330-337, 1996.
Hua et al., Mol. Vis., 15:1476-1484, 2009.
Hube et al., Infect. Immun., 65:3529-3538, 1997.
Hube et al., Mol. Microbiol., 14:87-99, 1994.
Irving et al., Sex Transm Infect, 74:334-8, 1998.
Jackson et al., Invest. Ophthalmol. Vis. Sci., 48:3559-3565, 2007.
Johansson et al., Apoptosis, 15:527-540, 2010.
Johnson et al., Antimicrob Agents Chemother, 43:1260-3, 1999.
Kamai, et al., 40^{th} Interscience Conference on: Antimicrobial Agents and Chemotherapy, Abstract #1080, p. 200, 2000.
Kauffman and Carver, Drugs, 35:539-549, 1997.
Kohler and Milstein, Eur. J. Immunol., 6:511-519, 1976.
Kohler and Milstein, Nature, 256:495-497, 1975.
Kubota et al., Nippon Ishinkin Gakkai Zasshi, 39:213-8, 1998.
Lee et al., Mycopathologia, 167:55-63, 2009.
Lin et al., Proc. Natl. Acad. Sci. USA, 97:1456-60, 2000.
Loaiza-Loeza et al., FEMS Immunol. Med. Microbiol., 56:212-222, 2009.
Lockhart et al., J. Clin. Microbiol., 34:767-77, 1996.
Lopez-Ribot et al., Antimicrob Agents Chemother., 42:2932-2937, 1998.
Lynch et al., J Med. Vet. Mycol, 34:337-9, 1996.
Markham et al., Antimicrob. Agents Chemotherapy 43:2404-2408, 1999.
Merrifield, Science, 232(4748):341-347, 1986.
Michalopoulos et al., Eur. J. Cardiothorac. Surg., 11:728-731, 1997.
Moran et al., Microbiology, 150:3363-3382, 2004.
Naglik et al., Microbiol. Mol. Biol. Rev., 67:400-428, 2003.
Naglik et al., Microbiol. Mol. Biol. Revs., 67:1092-2172, 2003.
Naglik et al., Microbiology, 154:3266-3280, 2008.
Oida et al., Chem. Pharm. Bull. (Tokyo), 48(5):694-707, 2000.
Pacheco-Rios et al., Arch. Med. Res., 28:229-232, 1997.
Rafiq et al., J. Biol. Chem., 281:19781-19792, 2006.
Redding et al., Clin. Infect. Dis. Feb., 18:240-2, 1994.
Remington's Pharmaceutical Sciences, 15^{th} Ed., 33:624-652, Mack Publishing Company, Easton, Pa., 1980.
Revankar et al. Am. J. Med. Jul., 105:7-11, 1998b.
Revankar et al., J. Clin. Microbiol., 36:153-156, 1998a.
Rex et al., In: Program and abstracts of the Infectious Diseases Society of America 34^{th} Annual Meeting Infectious Diseases Society of America, Washington, D.C., 74, p 49, 1996.
Sanglard, et al., Antimicrob. Agents and Chemother., 39:2378-2386, 1995.
Schroppel et al., J. Clin. Microbiol., 32:2646-2654, 1994.
Shigematsu et al., Jpn. J. Med. Mycol., 22, 195-201, 1981.
Staib et al., Mol. Microbiol., 32:533-546, 1999.
Staib et al., Proc. Natl. Acad. Sci. USA, 97:6102-6107, 2000.
Stewart and Young, In: Solid Phase Peptide Synthesis, 2d. ed., Pierce Chemical Co., 1984.
Takeda et al., 40th Interscience Conference on Antimicrobial Agents and Chemotherapy, Abstract #1080, p. 198, 2000.
Tam et al., J. Am. Chem. Soc., 105:6442, 1983.
Vanden Bossche et al., Med. Mycol., 36(1):119-28, 1998.
Vazquez et al., J. Infect. Dis., 170:1566-9, 1994.
Villar et al., Infect. Immun., 75:2126-2135, 2007.
Wawrzynczak & Thorpe, In: Immunoconjugates, Antibody Conjugates In Radioimaging And Therapy Of Cancer, Vogel (Ed.), NY, Oxford University Press, 28, 1987.
White and Agabian, J. Bacteriol., 177:5215-5221, 1995.
White et al., Clinical Microbiology Reviews, 11:382-402, 1998.
White et al., Genitorin Med., 73:475-6, 1997.
White, Antimicrob Agents Chemother., 41:1482-7, 1997.
Yotsuji et al., Antimicrob Agents Chemother, 41:30-4, 1997.

What is claimed is:

1. A method of inhibiting a secreted aspartic protease (SAP) cleavage of a target substrate comprising contacting said SAP with a peptide comprising at least four residues and having the formula:
P₂-P₁-P_{1′}-P_{2′}
wherein P₁, P₂, and P_{1′}, can be any residue, and P_{2′} is a negatively-charged residue.

2. The method of claim 1, wherein said peptide is 4-25 residues in length.

3. The method of claim 1, wherein said P_{2′} negatively-charged residue is aspartic acid, glutamic acid, phosphoric acid or sulfonic acid.

4. The method of claim 1, wherein said peptide comprises the sequence:
P₂-P₁-*-P_{1′}-P_{2′}
wherein -*- indicates modification of the peptide bond into a transition state analog.

5. The method of claim 1, wherein said peptide comprises the sequence SHLPS(E/D)FT.

6. The method of claim 1, wherein said peptide comprises the sequence SHLP*S(E/D)FT.

7. The method of claim 1, wherein said peptide comprises an XGY motif, wherein X is positively-charged residue, and Y is a negatively-charged residue.

8. The method of claim 7, wherein said peptide comprises the sequence RGD-SHLPS(E/D)FT or SHLPS(E/D)FT-RGD.

9. The method of claim 7, wherein said peptide comprises the sequence RGD-SHLP*S(E/D)FT or SHLP*S(E/D)FT-RGD, wherein * indicates modification of the peptide bond into a transition state analog.

10. The method of claim 1, wherein said SAP is SAP4, SAP5 or SAP6.

11. The method of claim 1, wherein said SAP is a pathogen SAP.

12. The method of claim 11, wherein said SAP is a yeast or fungus.

13. The method of claim 12, wherein said yeast is a Candida species or Aspergillus species.

14. The method of claim 13, wherein said Candida species is C. albicans.

15. The method of claim 13, wherein said Candida species a Candida tropicalis, Candida dubliniensis and Candida glabrata.

16. A peptide comprising at least four residues and having the formula:
P₂-P₁-*-P_{1′}-P_{2′}
wherein P₁, P₂ and P_{1′}, can be any residue, and P_{2′} is a negatively-charged residue, and -*- indicates modification of the peptide bond into a transition state analog.

17. The peptide of claim 16, wherein said peptide is 4-25 residues in length.

18. The peptide of claim 16, wherein said P_{2′} negatively-charged residue is aspartic acid, glutamic acid, phosphoric acid or sulfonic acid.

19. The peptide of claim 16, wherein said peptide comprises the sequence SHLP*S(E/D)FT.

20. The peptide of claim 16, wherein said peptide further comprises an XGY motif, wherein X is a positively-charged residue, and Y is a negatively-charged residue.

21. The peptide of claim 20, wherein said peptide comprises the sequence RGD-SHLP*S(E/D)FT or SHLP*S(E/D)FT-RGD.

22. The peptide of claim 20, wherein said peptide is linked to Integrilin®.

23. The peptide of claim 16, wherein said peptide is linked to a drug.

24. The peptide of claim 23, wherein said drug is an anti-fungal agent.

25. The peptide of claim 23, wherein said drug is a transition state inhibitor.

26. A method of inhibiting a fungal infection in a subject comprising administering to said subject a XGY motif peptide, wherein X is a positively-charged residue, and Y is a negatively-charged residue.

27. The method of claim 26, wherein said peptide is 4-25 residues in length.

28. The method of claim 26, wherein XGY motif peptide is linked to a second peptide having the formula:
P₂-P₁-*-P_{1′}-P_{2′}
wherein P₁, P₂, and P_{1′}, can be any residue, and P_{2′} is a negatively charged residue, and -*- indicates modification of the peptide bond into a transition state analog.

29. The method of claim 28, wherein said P_{2′} negatively-charged residue is aspartic acid, glutamic acid, phosphoric acid or sulfonic acid.

30. The method of claim 28, wherein said second peptide comprises the sequence SHLP*S(E/D)FT.

31. The method of claim 26, wherein said fungal infection is caused by a Candida species or Aspergillus species.

32. The method of claim 26, wherein said XGY motif comprises RGD.

33. The method of claim 26, wherein said RGD motif comprises RGDS.

34. The method of claim 26, wherein said XGY motif peptide is comprised in Integrilin®.

35. The method of claim 26, wherein said subject is a human subject.

36. The method of claim 26, wherein said peptide is linked to an anti-fungal agent.

37. A method of inhibiting a fungal infection in a subject comprising administering to said subject an antibody that binds immunologically to an XGY motif in a secreted aspartic protease, wherein X is a positively-charged residue, and Y is a negatively-charged residue.

38. The method of claim 37, wherein the motif is RGD.

39. The method of claim 38, wherein the motif is RGDS.

40. The method of claim 37, wherein said fungal infection is caused by a Candida species or Aspergillus species.


SEQUENCE LISTING (44 sequences)

SEQ ID NO: 1 - protein, 418 aa, Candida albicans
MFLKNILSVLAFALLIDAAPVKRSPGFVTLDFNVKRSLVDPDDPTVESKRSPLFLDLDPTQIPVDDTGRNDGVDKRGPVAVKLDNEIITYSADITVGSNNQKLSVIVDTGSSDLWIPDSKAICIPKWRGDRGDFCKNNGSYSPAASSTSKNLNTRFEIKYADGSYAKGNLYQDTVGIGGASVKNQLFANVWSTSAHKGILGIGFQTNEATRTPYDNLPISLKKQGIIAKNAYSLFLNSPEASSGQIIFGGIDKAKYSGSLVELPITSDRTLSVGLRSVNVMGRNVNVNAGVLLDSGTTISYFTPSIARSIIYALGGQVHFDSAGNKAYVADCKTSGTVDFQFDKNLKISVPASEFLYQLYYTNGKPYPKCEIRVRESEDNILGDNFMRSAYIVYDLDDKKISMAQVKYTSESNIVAIN

SEQ ID NO: 2 - protein, 417 aa, Candida albicans
MFLQNILSVLAFALLIDAAPVKRSTGFVTLDFNVKRSLVDPKDPTVEVKRSPLFLDIEPTEIPVDDTGRNDVGKRGPVAVKLDNEIITYSADITIGSNNQKLSVIVDTGSSDLWVPDSNAVCIPKWPGDRGDFCKNNGSYSPAASSTSKNLNTPFEIKYADGSVAQGNLYQDTVGIGGVSVRDQLFANVRSTSAHKGILGIGFQSNEATRTPYDNLPITLKKGQIISKNAYSLFLNSPEASSGQIISGGIDKAKYSGSLVDLPITSDRTLSVGLRSVNVMGQNVNVNAGVLLDSGTTISYFTPNIARSIIYALGGQVHYDSSGNEAYVADCKTSGTVDFQFDRNLKISVPASEFLYQLYYTNGEPYPKCEIRVRESEDNILGDNFMRSAYTVYDLDDRKISMAQVKYTSQSNIVAIN

SEQ ID NO: 3 - protein, 418 aa, Candida albicans
MFLKNILSVLAFALLIDAAPVKRSPGFVTLDFNVKRSLVDPDDPTVEAKRSPLFLEFTPSEFPVDETGRDGDVDKRGPVAVTLHNEAITYTADITVGSDNQKLNVIVDTGSSDLWIPDSNVICIPKWRGDKGDFCKSAGSYSPASSRTSQNLNTRFDIKYGDGSYAKGKLYKDTVGIGGVSVRDQLFANVWSTSARKGILGIGFQSGEATEFDYDNLPISLRNQGIIGKAAYSLYLNSAEASTGQIIFGGIDKAKYSGSLVDLPITSEKKLTVGLRSVNVRGRNVDANTNVLLDSGTTISYFTRSIVRNILYAIGAQMKFDSAGNKVYVADCKTSGTIDFQFGNNLKISVPVSEFLFQTYYTSGKPFRKCEVRIRESEDNILGDNFLRSAYVVYNLDDKKISMAPVKYTSESDIVAIN

SEQ ID NO: 4 - protein, 326 aa, Candida albicans
IGDEPLENYLDTEYFGTIGIGTPAQDFTVIFDTGSSNLWVPSVYCSSLACSDHNQFNPDDSSTFEATSQELSITYGTGSMTGILGYDTVQVGGISDTNQIFGLSETEPGSFLYYAPFDGILGLAYPSISASGATPVFDNLWDQGLVSQDLFSVYLSSNDDSGSVVLLGGIDSSYYTGSLNWVPVSVEGYWQITLDSITMDGETIACSGGCQAIVDTGTSLLTGPTSAIANIQSDIGASENSDGEMVISCSSIDSLPDIVFTIDGVQYPLSPSAYILQDDDSCTSGFEGMDVPTSSGELWILGDVFIRQYYTVFDRANNKVGLAPVA

SEQ ID NO: 5 - protein, 30 aa, Candida albicans
DTGSSNLWVPSVYCSSLACSDHNQFNPDDS

SEQ ID NO: 6 - protein, 39 aa, Candida albicans
DTGSSDLWVPDSNAVCIPKWPGDRGDFCKNNGSYSPAAS

SEQ ID NO: 7 - protein, 39 aa, Candida albicans
DTGSSDLWVPDSNVICIPKWRGDKGDFCKSAGSYSPASS

SEQ ID NO: 8 - protein, 39 aa, Candida albicans
DTGSSDLWVPDSKAICIPKWRGDRGDFCKNNGSYSPAAS

SEQ ID NO: 9 - protein, 5 aa, Candida albicans
MVLSA

SEQ ID NO: 10 - protein, 7 aa, Candida albicans
ADKGNVK

SEQ ID NO: 11 - protein, 7 aa, Candida albicans
GKVGGHA

SEQ ID NO: 12 - protein, 7 aa, Candida albicans
AEYGAEA

SEQ ID NO: 13 - protein, 7 aa, Candida albicans
GHAAEYG

SEQ ID NO: 14 - protein, 7 aa, Candida albicans
AEALERM

SEQ ID NO: 15 - protein, 6 aa, Candida albicans
AALTKA

SEQ ID NO: 16 - protein, 8 aa, Candida albicans
VEHLDDLP

SEQ ID NO: 17 - protein, 7 aa, Candida albicans
TKAVEHL

SEQ ID NO: 18 - protein, 8 aa, Candida albicans
DDLPGALS

SEQ ID NO: 19 - protein, 5 aa, Candida albicans
ALSEL

SEQ ID NO: 20 - protein, 7 aa, Candida albicans
SDHAHKL

SEQ ID NO: 21 - protein, 6 aa, Candida albicans
HAHKLR

SEQ ID NO: 22 - protein, 7 aa, Candida albicans
VDPVNFK

SEQ ID NO: 23 - protein, 6 aa, Candida albicans
LASHLP

SEQ ID NO: 24 - protein, 7 aa, Candida albicans
SDFTPAV

SEQ ID NO: 25 - protein, 4 aa, Candida albicans
MLTA

SEQ ID NO: 26 - protein, 6 aa, Candida albicans
EEKAAV

SEQ ID NO: 27 - protein, 6 aa, Candida albicans
KVDEVG

SEQ ID NO: 28 - protein, 8 aa, Candida albicans
GEALGRLL

SEQ ID NO: 29 - protein, 8 aa, Candida albicans
VYPWYQRF

SEQ ID NO: 30 - protein, 9 aa, Candida albicans
FESFGDLST

SEQ ID NO: 31 - protein, 6 aa, Candida albicans
FGDLST

SEQ ID NO: 32 - protein, 9 aa, Candida albicans
ADAVMNNPK

SEQ ID NO: 33 - protein, 7 aa, Candida albicans
KAHGKKV

SEQ ID NO: 34 - protein, 6 aa, Candida albicans
LDSFSN

SEQ ID NO: 35 - protein, 7 aa, Candida albicans
KGTFAAL

SEQ ID NO: 36 - protein, 8 aa, Candida albicans
SELHCDKL

SEQ ID NO: 37 - protein, 8 aa, Candida albicans
FAALSELH

SEQ ID NO: 38 - protein, 9 aa, Candida albicans
CDKLHVDPE

SEQ ID NO: 39 - protein, 8 aa, Candida albicans
HCDKLHVD

SEQ ID NO: 40 - protein, 8 aa, Candida albicans
PENFKLLG

SEQ ID NO: 41 - protein, 6 aa, Candida albicans
LARNFG

SEQ ID NO: 42 - protein, 6 aa, Candida albicans
KEFTPV

SEQ ID NO: 43 - protein, 8 aa, Candida albicans
  MISC_FEATURE (6)..(6): X = E or D
SHLPSXFT

SEQ ID NO: 44 - protein, 6 aa, Candida albicans
RGDKDG